(19) United States
(12) Patent Application Publication
(10) Pub. No.: US 2026/0217703 A1
(43) Pub. Date: Jul. 30, 2026

(54) INHIBITORS OF a-HEMOL YSIN OF STAPHYLOCOCCUS AUREUS

(71) Applicants: HELMHOLTZ-ZENTRUM FÜR INFEKTIONSFORSCHUNG GMBH, Braunschweig (DE); LEAD DISCOVERY CENTER GMBH, Dortmund (DE)

(72) Inventors: Mark Brönstrup, Braunschweig (DE); Raffaella Dilucrezia, Dortmund (DE)

(21) Appl. No.: 19/142,112

(22) PCT Filed: Dec. 19, 2023
(86) PCT No.: PCT/EP2023/086574

§ 371 Date: Jun. 20, 2025

(30) Foreign Application Priority Data
Dec. 21, 2022 (EP) 22215257.1

(51) Int. Cl. C07D 471/04; A61K 31/4985; A61K 31/538; C07D 519/00

(52) U.S. Cl. CPC C07D 471/04; A61K 31/4985; A61K 31/538; C07D 519/00

(57) ABSTRACT

The present invention relates to novel inhibitors of α-hemolysin of formula (I) and the use thereof for the prophylaxis and treatment of infections caused by Staphylococcus aureus; especially S. aureus lung infections.

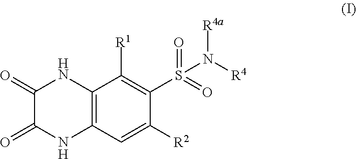

The present invention provides novel inhibitors of α-hemolysin and the use thereof for the prophylaxis and treatment of infections caused by Staphylococcus aureus; especially S. aureus lung infections.

Hospital-acquired bacterial pneumonia is the most frequent nosocomial infection. It is classified into two categories: HAP, which develops in hospitalized patients after 48 h of admission, and does not require artificial ventilation at the time of diagnosis, and VAP, which occurs in patients who have received mechanical ventilation for at least 48 h. Both types have a high mortality rate (>20%) in spite of adequate antibiotic therapy and require considerable health resources. S. aureus is among the most common pathogens associated with hospital acquired pneumonia worldwide. Treatment of these infections has become more challenging because of the global emergence of S. aureus strains resistant to commonly used antibiotics. In developed countries such as the USA, MRSA strains are a major problem in hospitals with up to one half of staphylococcal pneumonia isolates classified as MRSA, resulting in mortality as high as 56%.

The Infectious Diseases Society of America (IDSA) and American Thoracic Society (ATS) recommended vancomycin or linezolid in patients with hospital- and ventilation-acquired pneumonia (HAP/VAP) when empiric coverage of MRSA is indicated. Vancomycin poorly penetrates into the lung parenchyma, and high serum levels are often required to achieve adequate lung levels for bacterial killing. Unfortunately, increasing serum vancomycin levels comes with the risk of nephrotoxicity. Furthermore, the increasing prevalence of S. aureus strains with elevated vancomycin MIC (1-2 μg/mL) is associated with significantly treatment failure. Linezolid is not bactericidal against S. aureus and is not suitable for all patients due to drug interactions and hematologic effects.

The limited effectiveness of available standard-of-care treatments poses increasing public health risks. Thus, an improvement of current treatment regimens is critically needed for patients with HAP/VAP caused by S. aureus. An improvement cannot be achieved by bacterial killing with antibiotics alone, but require pre-emptive or adjunctive therapies that prevent or ameliorate the disease pathology on the host side. A highly promising approach, validated by preclinical and clinical data, is to block S. aureus' key virulence factor Hla and hence interfere with the capacity of S. aureus to colonize the lungs, thereby halting pathogenesis until the host immune response or antibiotics kill the bacteria.

It has therefore been an object of the present invention to provide novel inhibitors of virulence factor Hla.

The present invention provides compounds of formula (I):

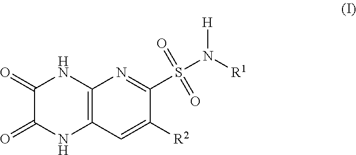

wherein
R² is hydrogen, halogen, OH, NO₂, CN or NH₂; or a C₁₋₄ alkyl group, a C₂₋₄ alkenyl group, a C₂₋₄ alkynyl group, a C₃₋₅ cycloalkyl group, an —O—C₃₋₅ cycloalkyl group, a C₄₋₈ alkylcycloalkyl group, or a C₁₋₄ heteroalkyl group; and
R¹ is an optionally substituted phenyl group; an optionally substituted naphthyl group; an optionally substituted heteroaryl group containing 1 or 2 rings and 5 to 10 ring atoms selected from O, S, N and C; an optionally substituted cycloalkyl aryl group comprising a phenyl group and a cycloalkyl group containing 5 or 6 ring atoms; an optionally substituted heterocycloalkyl aryl group comprising a phenyl group and a heterocycloalkyl group containing 5 or 6 ring atoms selected from O, S, B, N and C; an optionally substituted cycloalkyl heteroaryl group comprising a heteroaryl group comprising 5 or 6 ring atoms selected from O, S, N and C and a cycloalkyl group containing 5 or 6 ring atoms; or an optionally substituted heterocycloalkyl heteroaryl group comprising a heteroaryl group comprising 5 or 6 ring atoms selected from O, S, N and C and a heterocycloalkyl group containing 5 or 6 ring atoms selected from O, S, N and C; or an optionally substituted cycloalkyl group containing 1 or 2 rings and 3 to 10 ring atoms;
or a solvate, a hydrate or a salt thereof.

Preferably, R² is H, F, Cl, Br, a methyl group, an ethyl group, an iso-propyl group, a —CF₃ group, a methoxy group, a—OCD₃ group, a —OCF₃ group, a cyclopropyl group, a —CN group, a—CD₃ group, a —CHF₂ group, a —CH₂F group, a —CH₂OH group, a —NHMe group, an —O-cyclopropyl group, an —O—CH₂CF₃ group, an ethoxy group, an —NHCH₂CH₂OH group, or a —NMe₂ group.

Moreover preferably, R² is H, F, Cl, Br, a methyl group, an ethyl group, an iso-propyl group, a —CF₃ group, a methoxy group, a —OCF₃ group, a—OCD₃ group, a cyclopropyl group, a —CN group, a—CD₃ group, a —CHF₂ group, a —CH₂F group, a —CH₂OH group, a —NHCH₂CH₂OH group or a —NMe₂ group.

More preferably, R² is H, F, Cl, Br, a methyl group, an ethyl group, an iso-propyl group, a methoxy group, a trifluoromethoxy group or a CD₃ group.

Further preferably, R² is a group of formula —C(CH₃)═CH₂.

Moreover preferably, R² is H, Br, a methyl group, an iso-propyl group, or a —C(CH₃)═CH₂ group.

Especially preferably, R² is a methyl group.

Further preferably, R¹ is an optionally substituted phenyl group; an optionally substituted naphthyl group; an optionally substituted heteroaryl group containing 1 or 2 rings and 5 to 10 ring atoms selected from O, S, N and C; an optionally substituted cycloalkyl aryl group comprising a phenyl group and a cycloalkyl group containing 5 or 6 ring atoms; an optionally substituted heterocycloalkyl aryl group comprising a phenyl group and a heterocycloalkyl group containing 5 or 6 ring atoms selected from O, S, N and C; an optionally substituted cycloalkyl heteroaryl group comprising a heteroaryl group comprising 5 or 6 ring atoms selected from O, S, N and C and a cycloalkyl group containing 5 or 6 ring atoms; or an optionally substituted heterocycloalkyl heteroaryl group comprising a heteroaryl group comprising 5 or 6 ring atoms selected from O, S, N and C and a heterocycloalkyl group containing 5 or 6 ring atoms selected from O, S, N and C.

Moreover preferably, R¹ is an optionally substituted phenyl group; an optionally substituted naphthyl group; an optionally substituted heteroaryl group containing 1 or 2 rings and 5 to 10 ring atoms selected from O, S, N and C or an optionally substituted cycloalkyl aryl group comprising a phenyl group and a cycloalkyl group containing 5 or 6 ring atoms.

Further preferably, R¹ is an optionally substituted phenyl group; an optionally substituted naphthyl group; or an optionally substituted heteroaryl group containing 1 or 2 rings and 5 to 10 ring atoms selected from O, S, N and C.

Moreover preferably, R¹ is an optionally substituted phenyl group; or an optionally substituted heteroaryl group containing 5 or 6 ring atoms selected from O, S, N and C.

Further preferably, R¹ has the following formula:

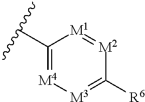

wherein
M¹ is N or CR⁷; M² is N or CR⁵; M³ is N or CR^{5a}; and M⁴ is N or CR^{7a};
R⁵, R^{5a}, R⁷ and R^{7a} are independently selected from hydrogen, halogen, CN, a C₁₋₄ alkyl group, a C₂₋₄ alkenyl group, a C₂₋₄ alkynyl group, or a C₁₋₄ heteroalkyl group; and R⁶ is halogen, CN, an alkyl group, an alkenyl group, an alkynyl group, a heteroalkyl group, a cycloalkyl group, a heterocycloalkyl group, an alkylcycloalkyl group, a heteroalkylcycloalkyl group, an aryl group, a heteroaryl group, an aralkyl group or a heteroaralkyl group; all of which groups may optionally be substituted; or
R⁶ is a group of formula —OR^{6a} or —NHR^{6a}, wherein R^{6a} is a cycloalkyl group, a heterocycloalkyl group, an alkylcycloalkyl group, a heteroalkylcycloalkyl group, an aryl group, a heteroaryl group, an aralkyl group or a heteroaralkyl group; all of which groups may optionally be substituted; or
R⁵ and R⁶ together are part of an optionally substituted phenyl group, an optionally substituted heteroaryl group containing 5 or 6 ring atoms selected from O, S, N and C, an optionally substituted cycloalkyl group containing 5 or 6 ring atoms or an optionally substituted heterocycloalkyl group containing 5 or 6 ring atoms selected from O, S, B, N and C (especially from O, S, N and C).

Preferably, only one of M¹, M², M³ and M⁴ is N.

Further preferably, none of M¹, M², M³ and M⁴ is N.

Moreover preferably, R⁷ is hydrogen or methyl; especially preferably, R⁷ is hydrogen.

Further preferably, R^{7a} is hydrogen.

Moreover preferably, R¹ is an optionally substituted phenyl group.

Further preferably, R¹ has the following formula:

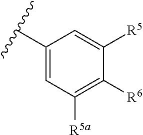

wherein
R⁵ and R^{5a} are independently selected from hydrogen, halogen, CN, a C₁₋₄ alkyl group, a C₂₋₄ alkenyl group, a C₂₋₄ alkynyl group, or a C₁₋₄ heteroalkyl group; and R⁶ is halogen, CN, an alkyl group, an alkenyl group, an alkynyl group, a heteroalkyl group, a cycloalkyl group, a heterocycloalkyl group, an alkylcycloalkyl group, a heteroalkylcycloalkyl group, an aryl group, a heteroaryl group, an aralkyl group or a heteroaralkyl group; all of which groups may optionally be substituted; or
R⁶ is a group of formula —OR^{6a} or —NHR^{6a}, wherein R^{6a} is a cycloalkyl group, a heterocycloalkyl group, an alkylcycloalkyl group, a heteroalkylcycloalkyl group, an aryl group, a heteroaryl group, an aralkyl group or a heteroaralkyl group; all of which groups may optionally be substituted; or
R⁵ and R⁶ together are part of an optionally substituted phenyl group, an optionally substituted heteroaryl group containing 5 or 6 ring atoms selected from O, S, N and C, an optionally substituted cycloalkyl group containing 5 or 6 ring atoms or an optionally substituted heterocycloalkyl group containing 5 or 6 ring atoms selected from O, S, B, N and C (especially from O, S, N and C).

Moreover preferably, R⁵ is hydrogen or methyl; especially hydrogen.

Further preferably, R¹ has the following formula:

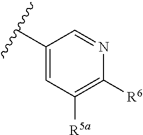

wherein
R^{5a} is selected from hydrogen, halogen, CN, a C₁₋₄ alkyl group, a C₂₋₄ alkenyl group, a C₂₋₄ alkynyl group, or a C₁₋₄ heteroalkyl group; and
R⁶ is halogen, CN, an alkyl group, an alkenyl group, an alkynyl group, a heteroalkyl group, a cycloalkyl group, a heterocycloalkyl group, an alkylcycloalkyl group, a heteroalkylcycloalkyl group, an aryl group, a heteroaryl group, an aralkyl group or a heteroaralkyl group; all of which groups may optionally be substituted; or
R⁶ is a group of formula —OR^{6a} or —NHR^{6a}, wherein R^{6a} is a cycloalkyl group, a heterocycloalkyl group, an alkylcycloalkyl group, a heteroalkylcycloalkyl group, an aryl group, a heteroaryl group, an aralkyl group or a heteroaralkyl group; all of which groups may optionally be substituted; or
R⁵ and R⁶ together are part of an optionally substituted phenyl group, an optionally substituted heteroaryl group containing 5 or 6 ring atoms selected from O, S, N and C, an optionally substituted cycloalkyl group containing 5 or 6 ring atoms or an optionally substituted heterocycloalkyl group containing 5 or 6 ring atoms selected from O, S, B, N and C (especially from O, S, N and C).

Further preferably, R^{5a} is hydrogen, Cl, Br, —CN, methyl, methoxy, —CF₃, —OCF₃, —NMe₂, —C≡CH, or —SO₂Me.

Further preferably, R^{5a} is hydrogen, Cl, Br, —CF₃, methyl or methoxy; especially, hydrogen, C₁, —CF₃ or methyl.

Moreover preferably, R^{5a} is hydrogen, Cl, Br, methyl or methoxy; especially, hydrogen, C₁ or methyl.

Further preferably, R⁶ is F, Cl, Br, CN, a C₁₋₆ alkyl group, a C₂₋₆ alkenyl group, a C₂₋₆ alkynyl group, a C₁₋₆ heteroalkyl group, an optionally substituted C₃₋₈ cycloalkyl group, an optionally substituted heterocycloalkyl group containing one or two rings and from 3 to 10 ring atoms selected from O, S, C and N, an optionally substituted phenyl group, an optionally substituted —CH₂-phenyl group, an optionally substituted heteroaryl group containing 5 or 6 to 10 ring atoms selected from O, S, N and C or an optionally substituted heterocycloalkyl aryl group comprising a phenyl group and a heterocycloalkyl group containing 4, 5 or 6 ring atoms selected from O, S, N and C.

Moreover preferably, R⁶ is CN, a C₂₋₆ alkenyl group, a C₂₋₆ alkynyl group, a C₂₋₆ heteroalkyl group, an optionally substituted C₃₋₈ cycloalkyl group, an optionally substituted heterocycloalkyl group containing one or two rings and from 3 to 10 ring atoms selected from O, S, C and N, an optionally substituted phenyl group, an optionally substituted —CH₂-phenyl group, an optionally substituted heteroaryl group containing 5 or 6 to 10 ring atoms selected from O, S, N and C or an optionally substituted heterocycloalkyl aryl group comprising a phenyl group and a heterocycloalkyl group containing 4, 5 or 6 ring atoms selected from O, S, N and C.

Further preferably, R⁶ is an optionally substituted C₃₋₈ cycloalkyl group, an optionally substituted heterocycloalkyl group containing one or two rings and from 3 to 10 ring atoms selected from O, S, C and N, an optionally substituted phenyl group, an optionally substituted —CH₂-phenyl group, an optionally substituted heteroaryl group containing 5 or 6 to 10 ring atoms selected from O, S, N and C or an optionally substituted heterocycloalkyl aryl group comprising a phenyl group and a heterocycloalkyl group containing 4, 5 or 6 ring atoms selected from O, S, N and C.

Moreover preferably, R⁶ is F, Cl, Br, CN, a C₁₋₆ alkyl group, a C₂₋₆ alkenyl group, a C₂-6 alkynyl group, a C₁₋₆ heteroalkyl group, an optionally substituted C₃₋₈ cycloalkyl group, an optionally substituted heterocycloalkyl group containing one or two rings and from 3 to 10 ring atoms selected from O, S, C and N, an optionally substituted phenyl group, an optionally substituted heteroaryl group containing 5 or 6 to 10 ring atoms selected from O, S, N and C or an optionally substituted heterocycloalkyl aryl group comprising a phenyl group and a heterocycloalkyl group containing 5 or 6 ring atoms selected from O, S, N and C.

Further preferably, R⁶ is a group of formula OR^{6a}, wherein R^{6a} is an optionally substituted C₃₋₈ cycloalkyl group, an optionally substituted heterocycloalkyl group containing one or two rings and from 3 to 10 ring atoms selected from O, S, C and N, an optionally substituted phenyl group, an optionally substituted heteroaryl group containing 5 or 6 to 10 ring atoms selected from O, S, N and C or an optionally substituted heterocycloalkyl aryl group comprising a phenyl group and a heterocycloalkyl group containing 5 or 6 ring atoms selected from O, S, N and C.

Moreover preferably, R⁶ is a group of formula NHR^{6a}, wherein R^{6a} is an optionally substituted 03-8 cycloalkyl group, an optionally substituted heterocycloalkyl group containing one or two rings and from 3 to 10 ring atoms selected from O, S, C and N, an optionally substituted phenyl group, an optionally substituted heteroaryl group containing 5 or 6 to 10 ring atoms selected from O, S, N and C or an optionally substituted heterocycloalkyl aryl group comprising a phenyl group and a heterocycloalkyl group containing 5 or 6 ring atoms selected from O, S, N and C.

Further preferably, R⁵ and R⁶ together are a group of formula —O—CH₂—O—, —O—CF₂—O— or —O—CH₂—CH₂—O—.

Moreover preferably, R⁶ is OCF₃.

Further preferably, R⁶ is an optionally substituted phenyl group or an optionally substituted heteroaryl group containing 5 or 6 to 10 ring atoms selected from O, S, N and C or an optionally substituted heterocycloalkyl aryl group comprising a phenyl group and a heterocycloalkyl group containing 5 or 6 ring atoms selected from O, S, N and C.

Moreover preferably, R⁶ is an optionally substituted phenyl group or an optionally substituted heteroaryl group containing 5 or 6 to 10 ring atoms selected from O, S, N and C.

Further preferably, R⁶ is F, Cl, Br, a C₁₋₆ alkyl group, a C₁₋₆ heteroalkyl group, an optionally substituted C₃₋₈ cycloalkyl group, an optionally substituted phenyl group, an optionally substituted heteroaryl group containing 5 or 6 to 10 ring atoms selected from O, S, N and C or an optionally substituted heterocycloalkyl aryl group comprising a phenyl group and a heterocycloalkyl group containing 4, 5 or 6 ring atoms selected from O, S, N and C.

According to a preferred embodiment, R⁶ is unsubstituted or substituted by 1, 2 or 3 substituents that are independently selected from halogen, CN, OH, NH₂, ═O, —P(═O)Me₂, COOH, CONH₂, a C₁₋₄ alkyl group, a C₂₋₄ alkenyl group, a C₂₋₄ alkynyl group, a C₁₋₄ heteroalkyl group, a C₃₋₇ cycloalkyl group, an —O—C₃₋₇ cycloalkyl group or a heterocycloalkyl group containing from 3 to 7 ring atoms selected from O, S, C and N.

According to a further preferred embodiment, R⁶ is unsubstituted or substituted by 1, 2 or 3 substituents that are independently selected from halogen, CN, COOH, CONH₂, a C₁₋₄ alkyl group, a C₂₋₄ alkenyl group, a C₂₋₄ alkynyl group, a C₁₋₄ heteroalkyl group, a C₃₋₇ cycloalkyl group or a heterocycloalkyl group containing from 3 to 7 ring atoms selected from O, S, C and N.

Especially preferably, the optionally substituted phenyl group or the optionally substituted heteroaryl group containing 5 or 6 to 10 ring atoms selected from O, S, N and C at R⁶ is unsubstituted or substituted by 1, 2 or 3 substituents that are independently selected from halogen (e.g., F or Cl), CN, COOH, a C₁₋₄ alkyl group, a C₂₋₄ alkenyl group, a C₂₋₄ alkynyl group, a C₁₋₄ heteroalkyl group, a C₃₋₇ cycloalkyl group or a heterocycloalkyl group containing from 3 to 7 ring atoms selected from O, S, C and N.

According to a further especially preferred embodiment, R⁶ is substituted by 1, 2 or 3 substituents that are independently selected from F, Cl, Br, CN, COOH, OH, NH₂, a —CF₃ group, a—CD₃ group, a —NMe₂ group, a methyl group, a methoxy group or a —CONH₂ group.

Especially preferred examples of substituents at R⁶ are F, C₁, methyl, methoxy, iso-propyl, cyclopropyl, and CN.

The most preferred compounds of the present invention are the compounds disclosed in the examples, or a salt, solvate or a hydrate thereof.

It is further preferred to combine the preferred embodiments of the present invention in any desired manner (e.g., any embodiment for R¹ may be combined with any embodiment of R²).

The expression alkyl refers to a saturated, straight-chain or branched hydrocarbon group that contains from 1 to 20 carbon atoms, preferably from 1 to 15 carbon atoms, especially from 1 to 10 (e.g., 1, 2, 3 or 4) carbon atoms, for example a methyl (Me, CH₃), ethyl, propyl, iso-propyl, n-butyl, iso-butyl, sec-butyl, tert-butyl, n-pentyl, iso-pentyl, n-hexyl, 2,2-dimethylbutyl or n-octyl group.

The expression C₁₋₆ alkyl refers to a saturated, straight-chain or branched hydrocarbon group that contains from 1 to 6 carbon atoms. The expression C₁₋₄ alkyl refers to a saturated, straight-chain or branched hydrocarbon group that contains from 1 to 4 carbon atoms. Examples are a methyl (Me), CF₃, CD₃, ethyl, propyl, iso-propyl, n-butyl, iso-butyl, sec-butyl or tert-butyl group.

The expressions alkenyl and alkynyl refer to at least partially unsaturated, straight-chain or branched hydrocarbon groups that contain from 2 to 20 carbon atoms, preferably from 2 to 15 carbon atoms, especially from 2 to 10 (e.g. 2, 3 or 4) carbon atoms, for example an ethenyl (vinyl), propenyl (allyl), iso-propenyl, butenyl, ethinyl, propinyl, butinyl, acetylenyl, propargyl, isoprenyl or hex-2-enyl group. Preferably, alkenyl groups have one or two (especially preferably one) double bond(s), and alkynyl groups have one or two (especially preferably one) triple bond(s).

Furthermore, the terms alkyl, alkenyl and alkynyl refer to groups in which one or more hydrogen atoms have been replaced by a halogen atom (preferably F or Cl) such as, for example, a 2,2,2-trichloroethyl, difluoromethyl, fluoromethyl or a trifluoromethyl group.

The expression heteroalkyl refers to an alkyl, alkenyl or alkynyl group as defined above in which one or more (preferably 1 to 8; especially preferably 1, 2, 3 or 4) carbon atoms have been replaced by an oxygen, nitrogen, phosphorus, boron, selenium, silicon or sulfur atom (preferably by an oxygen, sulfur or nitrogen atom) or by a SO or a SO₂ group. The expression heteroalkyl furthermore refers to a carboxylic acid or to a group derived from a carboxylic acid, such as, for example, acyl, acylalkyl, alkoxycarbonyl, acyloxy, acyloxyalkyl, carboxyalkylamide or alkoxycarbonyloxy. Furthermore, the term heteroalkyl refers to groups in which one or more hydrogen atoms have been replaced by a halogen atom (preferably F or Cl).

Preferably, a heteroalkyl group contains from 1 to 12 carbon atoms and from 1 to 8 heteroatoms selected from oxygen, nitrogen and sulfur (especially oxygen and nitrogen). Especially preferably, a heteroalkyl group contains from 1 to 6 (e.g. 1, 2, 3 or 4) carbon atoms and 1, 2, 3 or 4 (especially 1, 2 or 3) heteroatoms selected from oxygen, nitrogen and sulfur (especially oxygen and nitrogen). The term C₁-C₆ heteroalkyl refers to a heteroalkyl group containing from 1 to 6 carbon atoms and 1, 2, 3 or 4 heteroatoms selected from O, S and/or N (especially 0 and/or N). The term C₂-C₆ heteroalkyl refers to a heteroalkyl group containing from 2 to 6 carbon atoms and 1, 2, 3 or 4 heteroatoms selected from O, S and/or N (especially 0 and/or N). The term C₁-C₄ heteroalkyl refers to a heteroalkyl group containing from 1 to 4 carbon atoms and 1, 2 or 3 heteroatoms selected from O, S and/or N (especially 0 and/or N).

Further preferably, the expression heteroalkyl refers to an alkyl group as defined above (straight-chain or branched) in which one or more (preferably 1 to 6; especially preferably 1, 2, 3 or 4) carbon atoms have been replaced by an oxygen, sulfur or nitrogen atom or a CO group; this group preferably contains from 1 to 6 (e.g. 1, 2, 3 or 4) carbon atoms and 1, 2, 3 or 4 (especially 1, 2 or 3) heteroatoms selected from oxygen, nitrogen and sulfur (especially oxygen and nitrogen); this group may preferably be substituted by one or more (preferably 1 to 6; especially preferably 1, 2, 3 or 4) fluorine, chlorine, bromine or iodine atoms or OH, ═O, SH, ═S, NH₂, ═NH, N₃, CN or NO₂ groups.

Examples of heteroalkyl groups are groups of formulae: R^{a}—O—Y^{a}—, R^{a}—S—Y^{a}—, R^{a}—SO—Y^{a}—, R^{a}—SO₂—Y^{a}—, R^{a}—N(R^{b})—SO₂—Y^{a}—, R^{a}—SO₂—N(R^{b})—Y^{a}—, R^{a}—N(R^{b})—Y^{a}—, R^{a}—CO—Y^{a}—, R^{a}—C(═NR^{d})—Y^{a}—, R^{a}—O—CO—Y^{a}—, R^{a}—CO—O—Y^{a}—, R^{a}—CO—N(R^{b})—Y^{a}—, R^{a}—N(R^{b})—CO—Y^{a}—, R^{a}—N(R^{b})—C(═NR^{d})—Y^{a}—, R^{a}—O—CO—N(R^{b})—Y^{a}—, R^{a}—N(R^{b})—CO—O—Y^{a}—R^{a}—N(R^{b})—CO—N(R^{c})—Y^{a}—, R^{a}—O—CO—O—Y^{a}—, R^{a}—N(R^{b})—C(═NR^{d})—N(R^{c})—Y^{a}—, R^{a}—CS—Y^{a}—, R^{a}—O—CS—Y^{a}—, R^{a}—CS—O—Y^{a}—, R^{a}—CS—N(R^{b})—Y^{a}—, R^{a}—N(R^{b})—CS—Y^{a}—, R^{a}—O—CS—N(R^{b})—Y^{a}—, R^{a}—N(R^{b})CS—O—Y^{a}—, R^{a}—N(R^{b})—CS—N(R^{c})—Y^{a}—, R^{a}—O—CS—O—Y^{a}—, R^{a}—S—CO—Y^{a}—, R^{a}—CO—S—Y^{a}—, R^{a}—S—CO—N(R^{b})—Y^{a}—, R^{a}—N(R^{b})—CO—S—Y^{a}—, R^{a}—S—CO—O—Y^{a}—, R^{a}—O—CO—S—Y^{a}—, R^{a}—S—CO—S—Y^{a}—, R^{a}—S—CS—Y^{a}—, R^{a}—CS—S—Y^{a}—, R^{a}—S—CS—N(R^{b})—Y^{a}—R^{a}—N(R^{b})—CS—S—Y^{a}—, R^{a}—S—CS—O—Y^{a}—, R^{a}—O—CS—S—Y^{a}—, wherein R^{a} being a hydrogen atom, a C₁-C₆ alkyl, a C₂-C₆ alkenyl or a C₂-C₆ alkynyl group; R^{b} being a hydrogen atom, a C₁-C₆ alkyl, a C₂-C₆ alkenyl or a C₂-C₆ alkynyl group; R^{c} being a hydrogen atom, a C₁-C₆ alkyl, a C₂-C₆ alkenyl or a C₂-C₆ alkynyl group; R^{d} being a hydrogen atom, a C₁-C₆ alkyl, a C₂-C₆ alkenyl or a C₂-C₆ alkynyl group and Y^{a} being a bond, a C₁-C₆ alkylene, a C₂-C₆ alkenylene or a C₂-C₆ alkynylene group, wherein each heteroalkyl group contains at least one carbon atom. Further, one or more hydrogen atoms of the above groups may be replaced by fluorine or chlorine atoms.

Specific examples of heteroalkyl groups are methoxy, trifluoromethoxy,—OCD₃, —OCF₃, ethoxy, n-propyloxy, isopropyloxy, butoxy, tert-butyloxy, methoxymethyl, ethoxymethyl, —CH₂CH₂OH, —CH₂OH, —SO₂Me, —NHAc, —CONH₂, methoxyethyl, 1-methoxyethyl, 1-ethoxyethyl, 2-methoxyethyl or 2-ethoxyethyl, methylamino, ethylamino, propylamino, isopropylamino, dimethylamino, diethylamino, isopropylethylamino, methylamino methyl, ethylamino methyl, diisopropylamino ethyl, methylthio, ethylthio, isopropylthio, enol ether, dimethylamino methyl, dimethylamino ethyl, acetyl, propionyl, butyryloxy, acetyloxy, methoxycarbonyl, ethoxycarbonyl, propionyloxy, acetylamino or propionylamino, carboxymethyl, carboxyethyl or carboxypropyl, N-ethyl-N-methylcarbamoyl or N-methylcarbamoyl. Further examples of heteroalkyl groups are nitrile (—CN), isonitrile, cyanate, thiocyanate, isocyanate, isothiocyanate and alkylnitrile groups.

The expression cycloalkyl refers to a saturated or partially unsaturated (for example, a cycloalkenyl group) cyclic group that contains one or more rings (preferably 1 or 2), and contains from 3 to 14 ring carbon atoms, preferably from 3 to 10 (especially 3, 4, 5, 6 or 7) ring carbon atoms. The expression cycloalkyl refers furthermore to groups in which one or more hydrogen atoms have been replaced by fluorine, chlorine, bromine or iodine atoms or by OH, ═O, SH, ═S, NH₂, ═NH, N₃ or NO₂ groups, thus, for example, cyclic ketones such as, for example, cyclohexanone, 2-cyclohexenone or cyclopentanone. Further specific examples of cycloalkyl groups are a cyclopropyl, cyclobutyl, cyclopentyl, spiro[4,5]decanyl, norbornyl, cyclohexyl, cyclopentenyl, cyclohexadienyl, decalinyl, bicyclo[4.3.0]nonyl, tetraline, cyclopentylcyclohexyl, fluorocyclohexyl or cyclohex-2-enyl group.

The expression heterocycloalkyl refers to a cycloalkyl group as defined above in which one or more (preferably 1, 2 or 3) ring carbon atoms have been replaced by an oxygen, nitrogen, silicon, boron, selenium, phosphorus or sulfur atom (preferably by an oxygen, sulfur or nitrogen atom) or a SO group or a SO₂ group. A heterocycloalkyl group has preferably 1 or 2 ring(s) containing from 3 to 10 (especially 3, 4, 5, 6 or 7) ring atoms (preferably selected from C, O, N and S). The expression heterocycloalkyl refers furthermore to groups that are substituted by fluorine, chlorine, bromine or iodine atoms or by OH, ═O, SH, ═S, NH₂, ═NH, N₃ or NO₂ groups. Examples are a piperidyl, prolinyl, imidazolidinyl, piperazinyl, morpholinyl (e.g. —N(CH₂CH₂)₂O), urotropinyl, pyrrolidinyl, tetrahydrothiophenyl, tetrahydropyranyl, tetrahydrofuryl or 2-pyrazolinyl group and also lactames, lactones, cyclic imides and cyclic anhydrides.

The expression alkylcycloalkyl refers to groups that contain both cycloalkyl and also alkyl, alkenyl or alkynyl groups in accordance with the above definitions, for example alkylcycloalkyl, cycloalkylalkyl, alkylcycloalkenyl, alkenylcycloalkyl and alkynylcycloalkyl groups. An alkylcycloalkyl group preferably contains a cycloalkyl group that contains one or two rings having from 3 to 10 (especially 3, 4, 5, 6 or 7) ring carbon atoms, and one or two alkyl, alkenyl or alkynyl groups (especially alkyl groups) having 1 or 2 to 6 carbon atoms.

The expression heteroalkylcycloalkyl refers to alkylcycloalkyl groups as defined above in which one or more (preferably 1, 2 or 3) carbon atoms have been replaced by an oxygen, nitrogen, silicon, boron, selenium, phosphorus or sulfur atom (preferably by an oxygen, sulfur or nitrogen atom) or a SO group or a SO₂ group. A heteroalkylcycloalkyl group preferably contains 1 or 2 rings having from 3 to 10 (especially 3, 4, 5, 6 or 7) ring atoms, and one or two alkyl, alkenyl, alkynyl or heteroalkyl groups (especially alkyl or heteroalkyl groups) having from 1 or 2 to 6 carbon atoms. Examples of such groups are alkylheterocycloalkyl, alkylheterocycloalkenyl, alkenylheterocycloalkyl, alkynylheterocycloalkyl, heteroalkylcycloalkyl, heteroalkylheterocycloalkyl and heteroalkylheterocycloalkenyl, the cyclic groups being saturated or mono-, di- or tri-unsaturated.

The expression aryl refers to an aromatic group that contains one or more rings containing from 6 to 14 ring carbon atoms, preferably from 6 to 10 (especially 6) ring carbon atoms. The expression aryl refers furthermore to groups that are substituted by fluorine, chlorine, bromine or iodine atoms or by OH, SH, NH₂, N₃ or NO₂ groups. Examples are the phenyl, naphthyl, biphenyl, 2-fluorophenyl, anilinyl, 3-nitrophenyl or 4-hydroxyphenyl group.

The expression heteroaryl refers to an aromatic group that contains one or more rings containing from 5 to 14 ring atoms, preferably from 5 to 10 (especially 5 or 6 or 9 or 10) ring atoms, comprising one or more (preferably 1, 2, 3 or 4) oxygen, nitrogen, phosphorus or sulfur ring atoms (preferably 0, S or N). The expression heteroaryl refers furthermore to groups that are substituted by fluorine, chlorine, bromine or iodine atoms or by OH, SH, N₃, NH₂ or NO₂ groups. Examples are pyridyl (e.g. 4-pyridyl), imidazolyl (e.g. 2-imidazolyl), phenylpyrrolyl (e.g. 3-phenylpyrrolyl), thiazolyl, isothiazolyl, 1,2,3-triazolyl, 1,2,4-triazolyl, oxadiazolyl, thiadiazolyl, indolyl, indazolyl, tetrazolyl, pyrazinyl, pyrimidinyl, pyridazinyl, 4-hydroxypyridyl (4-pyridonyl), 3,4-hydroxypyridyl (3,4-pyridonyl), oxazolyl, isoxazolyl, triazolyl, tetrazolyl, isoxazolyl, indazolyl, indolyl, benzimidazolyl, benzoxazolyl, benzisoxazolyl, benzthiazolyl, pyridazinyl, quinolinyl, isoquinolinyl, pyrrolyl, purinyl, carbazolyl, acridinyl, pyrimidyl, 2,3′-bifuryl, pyrazolyl (e.g. 3-pyrazolyl) and isoquinolinyl groups.

The expression aralkyl refers to groups containing both aryl and also alkyl, alkenyl, alkynyl and/or cycloalkyl groups in accordance with the above definitions, such as, for example, arylalkyl, arylalkenyl, arylalkynyl, arylcycloalkyl, arylcycloalkenyl, alkylaryl-cycloalkyl and alkylarylcycloalkenyl groups. Specific examples of aralkyls are toluene, xylene, mesitylene, styrene, benzyl chloride, o-fluorotoluene, 1H-indene, tetraline, dihydronaphthalene, indanone, phenylcyclopentyl, cumene, cyclohexylphenyl, fluorene and indane. An aralkyl group preferably contains one or two aromatic ring systems (especially 1 or 2 rings), each containing from 6 to 10 carbon atoms and one or two alkyl, alkenyl and/or alkynyl groups containing from 1 or 2 to 6 carbon atoms and/or a cycloalkyl group containing 5 or 6 ring carbon atoms.

The expression heteroaralkyl refers to groups containing both aryl and/or heteroaryl groups and also alkyl, alkenyl, alkynyl and/or heteroalkyl and/or cycloalkyl and/or heterocycloalkyl groups in accordance with the above definitions. A heteroaralkyl group preferably contains one or two aromatic ring systems (especially 1 or 2 rings), each containing from 5 or 6 to 9 or 10 ring atoms (preferably selected from C, N, O and S) and one or two alkyl, alkenyl and/or alkynyl groups containing 1 or 2 to 6 carbon atoms and/or one or two heteroalkyl groups containing 1 to 6 carbon atoms and 1, 2 or 3 heteroatoms selected from O, S and N and/or one or two cycloalkyl groups each containing 5 or 6 ring carbon atoms and/or one or two heterocycloalkyl groups, each containing 5 or 6 ring atoms comprising 1, 2, 3 or 4 oxygen, sulfur or nitrogen atoms.

Examples are arylheteroalkyl, arylheterocycloalkyl, arylheterocycloalkenyl, arylalkylheterocycloalkyl, arylalkenylheterocycloalkyl, arylalkynylheterocycloalkyl, arylalkylheterocycloalkenyl, heteroarylalkyl, heteroarylalkenyl, heteroarylalkynyl, heteroarylheteroalkyl, heteroarylcycloalkyl, heteroarylcycloalkenyl, heteroaryl-heterocycloalkyl, heteroarylheterocycloalkenyl, heteroarylalkylcycloalkyl, heteroaryl-alkylheterocycloalkenyl, heteroarylheteroalkylcycloalkyl, heteroarylheteroalkyl-cycloalkenyl and heteroarylheteroalkylheterocycloalkyl groups, the cyclic groups being saturated or mono-, di- or tri-unsaturated. Specific examples are a tetrahydroisoquinolinyl, benzoyl, phthalidyl, 2- or 3-ethylindolyl, 4-methylpyridino, 2-, 3- or 4-methoxyphenyl, 4-ethoxyphenyl, 2-, 3- or 4-carboxyphenylalkyl group.

As already stated above, the expressions cycloalkyl, heterocycloalkyl, alkylcycloalkyl, heteroalkylcycloalkyl, aryl, heteroaryl, aralkyl and heteroaralkyl also refer to groups that are substituted by fluorine, chlorine, bromine or iodine atoms or by OH, ═O, SH, ═S, NH₂, ═NH, N₃ or NO₂ groups.

The term halogen refers to F, Cl, Br or I.

The term “optionally substituted” refers to a group which is unsubstituted or substituted by one or more (especially by one, two or three; preferably by one or two; especially preferably by one) substituents. If a group comprises more than one substituent, these substituents are independently selected, i.e., they may be the same or different.

Examples for substituents are fluorine, chlorine, bromine and iodine and OH, SH, NH₂, ═O, —SO₃H, —SO₂NH₂, —COOH, —COOMe,—COOEt, CH₂OH, —COMe (Ac), —NHSO₂Me, —SO₂NMe₂, —CH₂NH₂, —NHAc, —SO₂Me, —CONH₂, —CN, —NHCONH₂, —NHC(NH)NH₂, —NOHCH₃, —N₃ and —NO₂ groups. Further examples of substituents are C₁-C₁₀ alkyl, C₂-C₁₀ alkenyl, C₂-C₁₀ alkynyl, C₁-C₁₀ heteroalkyl, C₃-C₁₈ cycloalkyl, C₁-C₁₇ heterocycloalkyl, C₄-C₂₀ alkylcycloalkyl, C₁-C₁₉ heteroalkylcycloalkyl, C₆-C₁₈ aryl, C₁-C₁₇ heteroaryl, C₇-C₂₀ aralkyl and C₁-C₁₉ heteroaralkyl groups; especially C₁-C₆ alkyl, C₂-C₆ alkenyl, C₂-C₆ alkynyl, C₁-C₆ heteroalkyl, C₃-C₁₀ cycloalkyl, C₁-C₉ heterocycloalkyl, C₄-C₁₂ alkylcycloalkyl, C₁-C₁₁ heteroalkylcycloalkyl, C₆-C₁₀ aryl, C₁-C₉ heteroaryl, C₇-C₁₂ aralkyl and C₁-C₁₁ heteroaralkyl groups, further preferably C₁-C₆ alkyl and C₁-C₆ heteroalkyl groups.

Preferred substituents are halogen atoms (e.g. F, Cl, Br) and groups of formula —OH, ═O, —O—C₁₋₆ alkyl (e.g. —OMe,—OCD₃,—OEt, —O-nPr, —O-iPr, —O-nBu, —O-iBu and —O-tBu), —NH₂, —NHC₁₋₆ alkyl, —N(C₁₋₆ alkyl)₂, —COOH, —COOMe,—COOEt, —CH₂OH, —CH₂NH₂, —CH₂CH₂—O—CH₃, —COMe, —NHSO₂Me, —PO(CH₃)₂, —SO₂NMe₂, —SO₃H, —SO₂NH₂, —CONH₂, —CH₂NH₂, —CN, —C₁₋₆ alkyl (e.g. -Me, -Et, -nPr, -iPr, -nBu, -iBu, -tBu and —CF₃), —SH, —S—CO—C₁₋₆ alkyl, —S—C₁₋₆ alkyl, —NHAc, —NO₂, —C≡CH, —CH═C(CH₃)₂, —CH═CHCH₂OCH₂CH₃, —NHCONH₂, —SO₂NMe₂, —SO₂Me, phenyl, cyclopropyl, —O— cyclopropyl, and heterocycloalkyl groups containing from 3 to 6 ring atoms selected from O, N and C (especially one nitrogen atom and from 3 to 6 ring atoms).

Further preferred substituents are F, Cl, Br, ═O, a C₁₋₄ alkyl group (such as Me, Et, CF₃, iPr, tBu), a O—C₁₋₄ alkyl group (such as OMe, OCD₃, OCHF₂, OCH₂F, OiPr, OCF₃), NH₂, OH, a NHC₁₋₄ alkyl group, a N(C₁₋₄ alkyl)₂ group (such as NMe₂), —CH₂OH,—COOEt, —COOMe, —SO₂Me, —CH₂NH₂, —CH₂OH, —SO₂NMe₂, —NHCOCH₃, —SCF₃,—OCH₂CH₂NMe₂, —CH₂CH₂OCH₃, —NHCONMe₂, —PO(CH₃)₂, —COMe, —CONH₂, —COOH, —CN, —C≡CH, —CH═C(CH₃)₂, —CH═CHCH₂OCH₂CH₃, a pyrrolidinyl group, a-N(CH₂CH₂)₂₀ group, and an azetidinyl group.

When an aryl, heteroaryl, cycloalkyl, alkylcycloalkyl, heteroalkylcycloalkyl, heterocycloalkyl, aralkyl or heteroaralkyl group contains more than one ring, these rings may be bonded to each other via a single or double bond or these rings may be annulated.

The rings of any cycloalkyl aryl group, heterocycloalkyl aryl group, cycloalkyl heteroaryl group and heterocycloalkyl heteroaryl group may be bonded to each other via a single or double bond or these rings may be annulated.

It should be appreciated that certain compounds of formula (I) may have tautomeric forms from which only one might be specifically mentioned or depicted in the following description, different geometrical isomers (which are usually denoted as cis/trans isomers or more generally as (E) and (Z) isomers) or different optical isomers as a result of one or more chiral carbon atoms (which are usually nomenclatured under the Cahn-Ingold-Prelog or R/S system). All these tautomeric forms, geometrical or optical isomers (as well as racemates and diastereomers) and polymorphous forms are included in the invention. Since the compounds of formula (I) may contain asymmetric C-atoms, they may be present either as achiral compounds, mixtures of diastereomers, mixtures of enantiomers or as optically pure compounds. The present invention comprises both all pure enantiomers and all pure diastereomers, and also the mixtures thereof in any mixing ratio.

According to a further embodiment of the present invention, one or more hydrogen atoms of the compounds of the present invention may be replaced by deuterium. Deuterium modification improves the metabolic properties of a drug with little or no change in its intrinsic pharmacology. Deuterium substitution at specific molecular positions improves metabolic stability, reduces formation of toxic metabolites and/or increases the formation of desired active metabolites. Accordingly, the present invention also encompasses the partially and fully deuterated compounds of formula (I). The term hydrogen also encompasses deuterium.

The therapeutic use of compounds according to formula (I), their salts (especially their pharmacologically acceptable salts), solvates and hydrates, respectively, as well as formulations and pharmaceutical compositions also lie within the scope of the present invention.

The present invention further provides pharmaceutical compositions comprising one or more compounds described herein or a salt (especially a pharmaceutically acceptable salt), solvate or hydrate thereof, optionally in combination with one or more carrier substances and/or one or more adjuvants.

The present invention further provides a compound or a pharmaceutical composition as described herein for use in the prophylaxis, decolonization and treatment of a Staphylococcus aureus infection; especially for use in the prophylaxis and treatment of pneumonia caused by Staphylococcus aureus.

The present invention moreover provides a compound or a pharmaceutical composition as described herein for the preparation of a medicament, especially for the prophylaxis, decolonization and treatment of a Staphylococcus aureus infection; especially for the prophylaxis and treatment of pneumonia caused by Staphylococcus aureus.

According to a further preferred embodiment, the present invention provides a method for prophylaxis, decolonization and/or treatment of a Staphylococcus aureus infection; especially for prophylaxis and/or treatment of pneumonia caused by Staphylococcus aureus in a subject which comprises administering to the subject an effective amount of a compound of formula (I), or a pharmaceutically acceptable salt thereof.

According to a moreover preferred embodiment, the present invention provides a method for prophylaxis, decolonization and/or treatment of a Staphylococcus aureus infection; especially for prophylaxis and/or treatment of pneumonia caused by Staphylococcus aureus in a subject which comprises administering to the subject an effective amount of a pharmaceutical composition comprising a compound of formula (I), or a pharmaceutically acceptable salt thereof.

The present invention also relates to pro-drugs which are composed of a compound of formula (I) and at least one pharmacologically acceptable protective group which will be cleaved off under physiological conditions, such as an alkoxy-, arylalkyloxy-, acyl-, acyloxymethyl group (e.g. pivaloyloxymethyl), an 2-alkyl-, 2-aryl- or 2-arylalkyl-oxycarbonyl-2-alkylidene ethyl group or an acyloxy group as defined herein, e.g. ethoxy, benzyloxy, acetyl or acetyloxy or, especially for a compound of formula (I), carrying a hydroxy group (—OH): a sulfate, a phosphate (—OPO3 or —OCH₂OPO3) or an ester of an amino acid.

Preferably, the present invention also relates to a prodrug, a biohydrolyzable ester, a biohydrolyzable amide, a polymorph, tautomer, stereoisomer, metabolite, N-oxide, biohydrolyzable carbamate, biohydrolyzable ether, physiologically functional derivative, atropisomer, or in vivo-hydrolysable precursor, diastereomer or mixture of diastereomers, chemically protected form, affinity reagent, complex, chelate and a stereoisomer of the compounds of formula (I).

Examples of pharmacologically acceptable salts of sufficiently basic compounds are salts of physiologically acceptable mineral acids like hydrochloric, hydrobromic, sulfuric and phosphoric acid; or salts of organic acids like methanesulfonic, p-toluenesulfonic, lactic, acetic, trifluoroacetic, citric, succinic, fumaric, maleic and salicylic acid. Further, a sufficiently acidic compound may form alkali or earth alkali metal salts, for example sodium, potassium, lithium, calcium or magnesium salts; ammonium salts; or organic base salts, for example methylamine, dimethylamine, trimethylamine, triethylamine, ethylenediamine, ethanolamine, choline hydroxide, meglumin, piperidine, morpholine, tris-(2-hydroxyethyl)amine, lysine or arginine salts; all of which are also further examples of salts of the compounds described herein.

The compounds described herein may be solvated, especially hydrated. The hydratization/hydration may occur during the process of production or as a consequence of the hygroscopic nature of the initially water-free compounds. The solvates and/or hydrates may e.g. be present in solid or liquid form.

In general, the compounds and pharmaceutical compositions described herein will be administered by using the known and acceptable modes known in the art.

For oral administration such therapeutically useful agents can be administered by one of the following routes: oral, e.g. as tablets, dragees, coated tablets, pills, semisolids, soft or hard capsules, for example soft and hard gelatine capsules, aqueous or oily solutions, emulsions, suspensions or syrups, parenteral including intravenous, intramuscular and subcutaneous injection, e.g. as an injectable solution or suspension, rectal as suppositories, by inhalation or insufflation, e.g. as a powder formulation, as microcrystals or as a spray (e.g. liquid aerosol), transdermal, for example via an transdermal delivery system (TDS) such as a plaster containing the active ingredient or intranasal. For the production of such tablets, pills, semisolids, coated tablets, dragees and hard, e.g. gelatine, capsules the therapeutically useful product may be mixed with pharmaceutically inert, inorganic or organic excipients as are e.g. lactose, sucrose, glucose, gelatine, malt, silica gel, starch or derivatives thereof, talc, stearinic acid or their salts, dried skim milk, and the like. For the production of soft capsules one may use excipients as are e.g. vegetable, petroleum, animal or synthetic oils, wax, fat, and polyols. For the production of liquid solutions, emulsions or suspensions or syrups one may use as excipients e.g. water, alcohols, aqueous saline, aqueous dextrose, polyols, glycerin, lipids, phospholipids, cyclodextrins, vegetable, petroleum, animal or synthetic oils. Especially preferred are lipids and more preferred are phospholipids (preferred of natural origin; especially preferred with a particle size between 300 to 350 nm) preferred in phosphate buffered saline (pH=7 to 8, preferred 7.4). For suppositories one may use excipients as are e.g. vegetable, petroleum, animal or synthetic oils, wax, fat and polyols. For aerosol formulations one may use compressed gases suitable for this purpose, as are e.g. oxygen, nitrogen and carbon dioxide. The pharmaceutically useful agents may also contain additives for conservation, stabilization, e.g. UV stabilizers, emulsifiers, sweetener, aromatizers, salts to change the osmotic pressure, buffers, coating additives and antioxidants.

In general, in the case of oral or parenteral administration to adult humans weighing approximately 80 kg, a daily dosage of about 1 mg to about 10,000 mg, preferably from about 5 mg to about 1,000 mg, should be appropriate, although the upper limit may be exceeded when indicated. The daily dosage can be administered as a single dose or in divided doses, or for parenteral administration, it may be given as continuous infusion or subcutaneous injection.

### EXAMPLES

### Abbreviations and Acronyms

Abbreviations and Acronyms used in the description of the chemistry and in the Examples that follow are:
AcOH acetic acid
aq. aqueous
br. broad
CDCl₃ deuterated chloroform
CHCl₃ chloroform
Cs₂CO₃ cesium carbonate
d doublet
DAD diode array detector
DCM dichloromethane
Dd doublet of doublets
DIPEA diisopropylethylamine
DMF dimethylformamide
DMSO dimethylsulfoxide
DMSO-d₆ deuterated dimethylsulfoxide
ES electrospray
Et₂O diethylether
EtOAc ethyl acetate
EtOH ethanol
FCS fetal calf serum
h hour
HBBS Hanks's Balanced Salt Solution
HEPES 4-(2-hydroxyethyl)-1-piperazineethanesulfonic acid
HPLC high performance liquid chromatography
H₂O water
H₂O₂ hydrogen peroxide
Hz Herz
K₂CO₃ potassium carbonate
LC liquid chromatography
LDH lactate dehydrogenase
m multiplet
MeCN acetonitrile
MeOH methanol
min minutes
MS mass spectrometry
NaHCO₃ sodium hydrogencarbonate
Na₂SO₄ sodium sulfate
NBS N-bromosuccinimide
NCS N-chlorosuccinimide
NH₃ ammonia
NH₄Cl ammonium chloride
(NH₄)₂CO₃ ammonium carbonate
NH₄HCO₃ ammonium bicarbonate
NMR nuclear magnetic resonance
PBS Phosphate Buffered Saline
Pd₂(dba)₃ Tris-(dibenzylidenaceton)-dipalladium(0)
Pd(PPh₃)₄ Tetrakis(triphenylphosphine)palladium(0)
pet-ether petroleum ether
POCl₃ phosphoryl chloride
prep-HPLC preparative high performance liquid chromatography
q quartet
rpm rounds per minute
r. t. room temperature
s singlet
sat. saturated
SQ single quadrupole
t triplet
t-BuOH tert-butanol
tBu₃PPdG2 Chloro[(tri-tert-butylphosphine)-2-(2-aminobiphenyl)]palladium(II)
THF tetrahydrofurane
TFA trifluoroacetic acid
TFAA trifluoroacetic acid anhydride
UPLC Ultra Performance Liquid Chromatography
Xantphos (9,9-Dimethyl-9H-xanthene-4,5-diyl)bis(diphenylphosphane)
Zn Zinc

### 1. Methods of Making the Compounds of Formula (I) of the Present Invention

In general, the compounds of formula (I) of the invention might be prepared by standard techniques known in the art, by known processes analogous thereto, and/or by the processes described herein, using starting materials which are either commercially available or producible according to conventional chemical methods. The particular processes to be utilized in the preparation of the compounds of formula (I) of this invention depend upon the specific compound desired. Such factors as the type of substitution at various locations of the molecule and the commercial availability of the starting materials play a role in the path to be followed and in the chosen reaction conditions for the preparation of the specific compounds of formula (I) of this invention. Those factors are readily recognized by one of ordinary skill in the art.

The following preparative methods are presented to aid the reader in the synthesis of the compounds of the present invention.

### 2. Experimental Procedures

### LC-MS Method

HPLC—electrospray mass spectra (HPLC ES-MS) were obtained using a Waters Acquity Ultra Performance Liquid Chromatography (UPLC) equipped with a SQ 3100 Mass detector spectrometer.
Column: Acquity UPLC BEH C18 1.7 μm, 2.1×50 mm
Flow: 0.500 mL/min
Eluents: A: H₂O with 0.05% formic acid and B: MeCN with 0.05% formic acid.
Gradient: elution from 5% to 100% B over 3.5 min with an initial hold of 0.5 min and a final hold at 100% B of 0.5 min. Total run time: 5 min.

The gradient described could be altered in function of the physico-chemical properties of the compound analysed and is in no way restrictive.

### Preparative HPLC Method

Preparative HPLC was performed using an Agilent System consisting of an Agilent Infinity 1260 Autosampler, an Agilent Infinity 1260 Binary Gradient Module, an Agilent 6120 Quadrupole Mass Detector and an Agilent Infinity 1260 DAD VL UVNisible Detector.

Column: XBridge® BEH Prep C18 5 μm, 19 mm×150 mm
Flow: 32 mL/min
Eluents: A: H₂O with 0.1% TFA and B: MeCN with 0.1% TFA.
General Gradient: elution from X % to Y % B over 20 min with an initial hold of 2 min and a final increase to 100% B over 2 min and hold at 100% B of 2 min followed by a 1 min gradient back to the initial composition. Total run time: 26 min. X=Y−30% where Y=concentration of elution for the above described LC-MS method.
Alternatively:
Column: Gemini NX C18 (250*10), 5μ
Flow: 6 mL/min
Eluents: A: 10 nM (NH₄)HCO₃ in H₂O and B: MeCN.
General Gradient: elution from X % to Y % B over 20 min with an initial hold of 2 min and a final increase to 100% B over 0.1 min and hold at 100% B of 3 min followed by a 3 min gradient back to the initial composition. Total run time: 26 min. X=Y−30% where Y=concentration of elution for the above described LC-MS method.

The gradient described could be altered in function of the physico-chemical properties of the compound analyzed and is in no way restrictive.

### NMR Methods

Proton (¹H) nuclear magnetic resonance (NMR) spectra were measured with a Bruker Avance III (500 MHz) spectrometer with residual protonated solvent (CHCl3δ7.26; MeOHδ3.30; DMSOδ2.49) as standard. The NMR data of the synthesized examples are in agreement with their corresponding structural assignments.

### 2.1 Experimental Examples of the Invention

### 2.1.1. Synthetic Methods

The sulphonyl chloride intermediates of formula A, necessary to generate the compounds of the invention were synthesized from commercially available 2-chloro-3-methyl-5-nitropyridine, 2-amino-3-nitro-6-chloropyridine, or 2,3-diamino-6-chloropyridine (4c) according to the scheme below, and were used crude in the sulfonamide formation reaction:

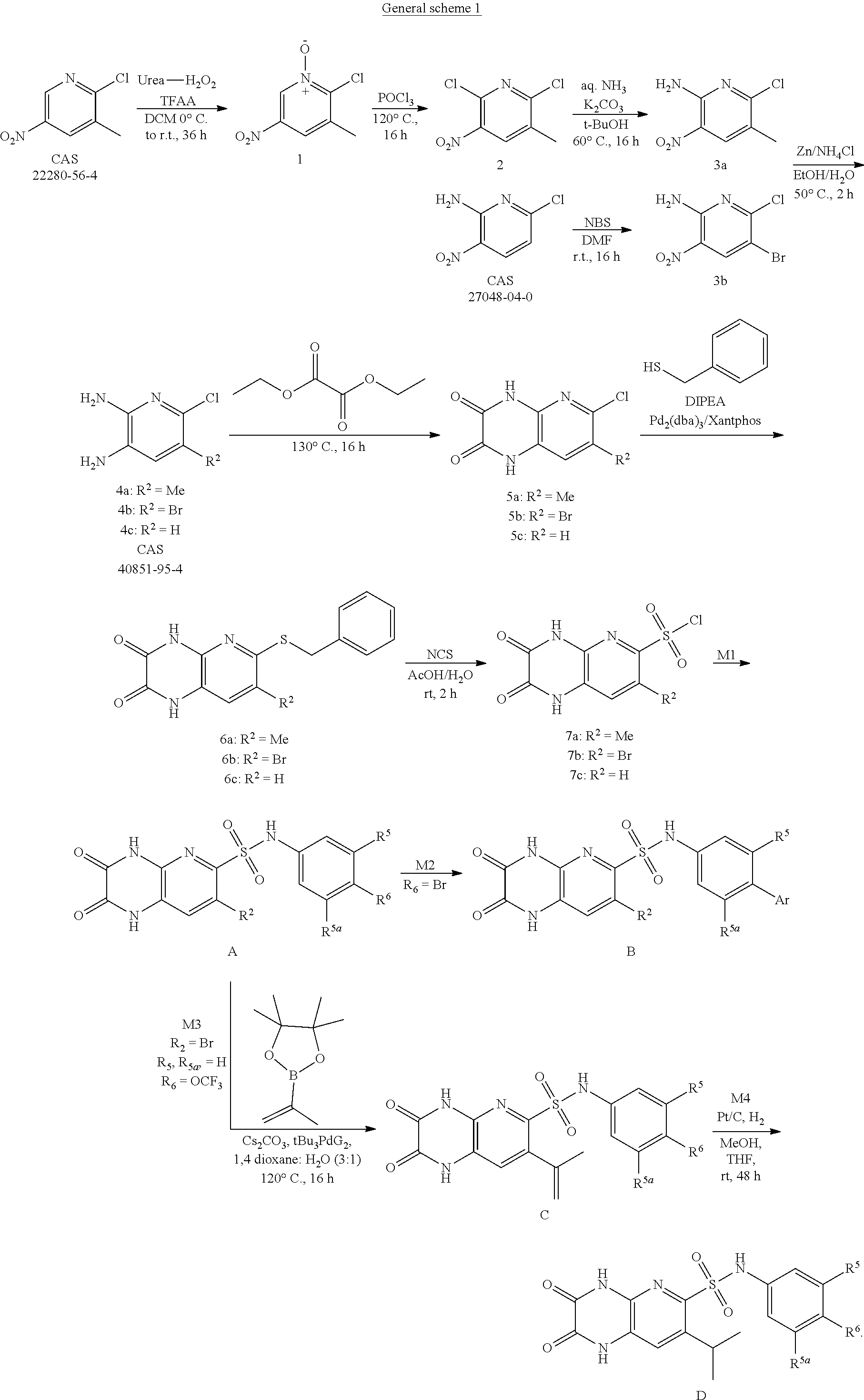

The majority of the compounds of the invention were synthesized according to General Scheme 1 described above, where compound 7 is the building block used for the following sulfonamide formation in the coupling step M1 with commercially available aniline derivatives to give compounds of formula A. When substituent R⁶ is a halogen, a further Suzuki coupling could be performed to yield biaryls of formula B, where R⁶ is an aryl or heretoaryl group, identified by —Ar in general scheme 1. Similarly, when substituent R² is a halogen, a further metal-catalyzed coupling reaction can be carried out to introduce an aryl, heteroaryl, or alkenyl group, as illustrated in scheme 1 for 2-isopropenylboronic acid pinacol ester to obtain compounds of formula C. Further reduction of compounds C leads to compounds of formula D. It should be apparent to a person skilled in the art that the sequence of the synthetic steps is dependent on starting materials availability and functional group compatibility and could vary from compound to compound. In particular, steps M1 and M2 could easily be reversed to obtain in a first instance a biarylaniline (such as compound 8) or a para-substituted dianiline intermediate, which could then be reacted with sulphonyl chlorides 7 to obtain the final compounds of formula B. Similar conditions as for described methods M1 and M2 can be applied.

The following specific examples are presented to illustrate the invention, but they should not be construed as limiting the scope of the invention in any way. In the tables listing the intermediates, the compounds might have characterization such as (M+H)⁺ mass spectrometry data, HPLC purity and/or NMR.

### 2.1.2. Preparation of Intermediate Compounds of Formula (A and B)

### 2-chloro-3-methyl-5-nitropyridine 1-oxide (1)

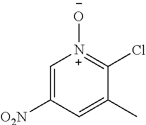

To a stirred solution of 2-chloro-3-methyl-5-nitropyridine (5 g, 29.07 mmol) and Urea-H₂O₂ (5.74 g, 61.04 mmol) in DCM (100 mL) at 0° C. was added TFAA (8.5 mL, 61.04 mmol) dropwise. The reaction mixture was allowed to warm up to r. t. and stirred for 36 h, upon which it was diluted with H₂O and extracted with DCM. The combined organic layer was dried over anhydrous Na₂SO₄, filtered and reduced in vacuo. The crude was purified by flash chromatography on silica gel using a gradient of MeOH in DCM to yield the desired compound 1 (5 g, 91%) as a dark green solid. ¹H NMR (400 MHz, DMSO-d₆) δ: 7.05 (dd, J=1.6, 0.8 Hz, 1H), 7.99 (d, J=0.8 Hz, 1H), 2.33 (s, 3H).

MS (ES) C₆H₅ClN₂O₃ requires: 188, found: 189 (M+H)⁺, 99%

### 2,6-dichloro-3-methyl-5-nitropyridine (2)

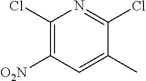

To a solution of compound 1 (10 g, 53.19 mmol) was added POCl₃ (50 mL) at r. t. and the mixture was stirred at 120° C. for 16 h. The reaction was cooled to 0° C., quenched with cold H₂O, and extracted with EtOAc. The combined organic layer was washed with sat. NaHCO₃ solution and brine, dried over anhydrous Na₂SO₄, filtered and reduced in vacuo. The crude was purified by flash chromatography on silica gel column, using a gradient of MeOH in DCM to yield the desired compound 2 (10 g, 91%) as a yellow solid.

MS (ES) C₆H₄C₁₂N₂O₂ requires: 206, found: 207 (M+H)⁺, 93%

### 6-chloro-5-methyl-3-nitropyridin-2-amine (3a)

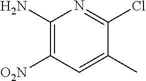

To a solution of compound 2 (4.8 g, 23.18 mmol) in t-BuOH (50 mL) were added K₂CO₃ (4.8 g, 34.70 mmol) and aq. NH₃ (7.88 mL, 115.90 mmol) and the mixture was stirred at 60° C. for 16 h. The reaction was cooled to r. t. and quenched with H₂O. The precipitated solid was filtered, washed with H₂O and dried under vacuum. The crude was purified by flash chromatography on silica gel, using a gradient of EtOAc in pet-ether to yield the desired compound 3a (2.4 g, crude) as a yellow solid, which was used in the following step without further purification.

MS (ES) C₆H₆ClN₃O₂ requires: 187, found: 188 (M+H)⁺, 55% For the synthesis of compound 25, a similar route as described for compound 9 was employed, starting from the 6-chloro-5-bromo-3-nitropyridin-2-amine 3b, which was prepared as follows:

### 6-chloro-5-methyl-3-nitropyridin-2-amine (3b)

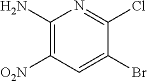 [SMILES: Nc1nc(Cl)c(Br)cc1[N+](=O)[O-]]

To a stirred solution of 6-chloro-3-nitropyridin-2-amine (1 g, 5.78 mmol) in DMF (10 mL) was added NBS (1.2 g, 6.93 mmol) at r. t. under argon atmosphere. The reaction mixture was then stirred at r. t. for 16 h, upon which the mixture was quenched with ice-H₂O and extracted with EtOAc. The combined organic layers were washed with brine, dried over anhydrous Na₂SO₄, filtered, and reduced in vacuo to yield the desired compound 3b (1.6 g, crude) as yellow solid, which was used in the following step without further purification.

¹H NMR (400 MHz, DMSO-d₆) δ: 8.63 (s, 1H), 8.32 (s, 2H).

MS (ES) C₅H₃BrClN₃O₂ requires: 250, 252, found: 251, 253 (M+H)⁺, 80%

### 6-chloro-5-methylpyridine-2,3-diamine (4a)

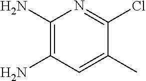

To a stirred solution of compound 3a (2.3 g, 12.29 mmol) in a mixture of MeOH (6 mL), H₂O (3 mL) and THF (6 mL) at r. t. Zn dust (6.4 g, 98.3 mmol) and NH₄Cl (6.5 g, 122 mmol) were added and the reaction was stirred at 40° C. for 2 h. The reaction mixture was filtered through celite and the celite bed was washed with 50% MeOH in THF. The filtrate was reduced in vacuo and the residue was partitioned between H₂O and 10% MeOH in DCM. The aqueous layer was extracted with 10% MeOH in DCM and the combined organic layer was reduced in vacuo. The crude was co-distilled with toluene to yield the desired compound 4a (1.8 g, crude) as a brown solid, which was used in the following step without further purification.

¹H NMR (500 MHz, DMSO-d₆) δ: 6.64 (s, 1H), 5.55 (s, 2H), 4.72 (s, 2H), 2.04 (s, 3H).

MS (ES) C₆H₈ClN₃ requires: 157, found: 158 (M+H)⁺, 84%

### 6-chloro-7-methyl-1,4-dihydropyrido[2,3-b]pyrazine-2,3-dione (5a)

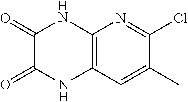

A mixture of compound 4a (1.8 g, 11.46 mmol) and diethyl oxalate (36 mL) was stirred at 130° C. for 16 h in a sealed tube. The reaction was cooled to r. t. and diluted with Et₂O. The resulting solid was filtered and dried under vacuum to yield the desired compound 5a (2 g, 82%) as a light brown solid.

MS (ES) C₈H₆ClN₃O₂ requires: 211, found: 212 (M+H)⁺, 96% 6-(benzylthio)-7-methyl-1,4-dihydropyrido[2,3-b]pyrazine-2,3-dione (6a)

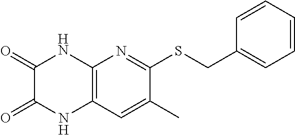 [SMILES: Cc1cc2[nH]c(=O)c(=O)[nH]c2nc1SCc1ccccc1]

To a degassed solution of compound 5a (500 mg, 2.36 mmol), phenylmethanethiol (0.32 mL, 2.59 mmol) and DIPEA (0.84 mL, 4.72 mmol) in dioxane (5 mL) were added, followed by Pd₂(dba)₃ (108 mg, 0.118 mmol) and Xantphos (136 mg, 0.236 mmol) at r. t. The resulting mixture was stirred at 150° C. for 3 h in the microwave. The reaction was cooled to r. t. and poured into cold H₂O. The resulting solid was filtered and dried in vacuo to yield the desired compound 6a (300 mg, 42%) as a light brown solid.

¹H NMR (400 MHz, DMSO-d₆) δ:12.36 (s, 1H), 11.91 (s, 1H), 7.50 (d, J=6.8 Hz, 2H), 7.28 (t, J=6.8 Hz, 2H), 7.23-7.20 (m, 2H), 4.42 (s, 2H), 2.13 (s, 3H).

MS (ES) C₁₅H₁₃N₃₀₂S requires: 299, found: 300 (M+H)⁺, 93% 6-(benzylthio)-7-methyl-1,4-dihydropyrido[2,3-b]pyrazine-2,3-dione (7a)

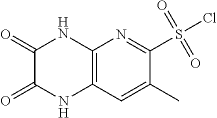 [SMILES: Cc1cc2[nH]c(=O)c(=O)[nH]c2nc1S(=O)(=O)Cl]

To a solution of compound 6a (400 mg, 1.33 mmol) in AcOH (4.5 mL) at 0° C. were added NCS (711 mg, 5.35 mmol) and H₂O (1 mL), upon which the reaction was allowed to warm up r. t. and stirred for 3 h. The crude mixture was reduced in vacuo to yield compound 7a (400 mg) as a brown solid, which was used in the following step without further purification.

MS (ES) C₈H₆ClN₃O₄S requires: 275, found: 276 (M+H)⁺, ~5%, MS (ES); sulfonic acid: C₈H₇N₃₀₅S requires: 257, found: 258 (M+H)⁺, 83%

### Compound 9—Synthesis According to Method 1 (M1) and Method 2 (M2)

### 2-chloro-4′-fluoro-[1,1′-biphenyl]-4-amine (8)—Synthesis According to Method 2 (M2)

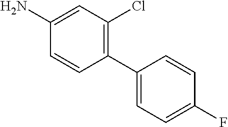 [SMILES: Nc1ccc(-c2ccc(F)cc2)c(Cl)c1]

To a degassed solution of 4-bromo-3-chloroaniline (200 g, 0.97 mol) were added (4-fluorophenyl)boronic acid (160 g, 1.16 mol) and Cs₂CO₃ (248 g, 2.91 mol) in a mixture of 1,4-dioxane (2 L) and H₂O (1 L). Pd(PPh₃)₄ (56 g, 0.04 mol) was added at r. t. The resulting mixture was stirred at 80° C. for 16 h, upon which the reaction was cooled to r. t., diluted with H₂O (1 L) and extracted with EtOAc (4×500 mL). The combined organic layer was washed with H₂O (2×150 mL), brine (250 mL), dried over anhydrous Na₂SO₄ and reduced in vacuo. The crude was purified by flash chromatography on silica gel, using a gradient of EtOAc in pet-ether to afford the desired compound 8 (207 g, 96%) as a brown solid.

¹H NMR (500 MHz, CDCl₃): δ 7.38 (dd, J=8.9, 5.5 Hz, 2H), 7.13-7.05 (m, 3H), 6.80 (d, J=2.4 Hz, 1H), 6.63 (dd, J=8.2, 2.4 Hz, 1H), 3.77 (br. m, 2H).

MS (ES) C₁₂H₉ClFN requires: 221, found: 222 (M+H)⁺, 68%

### N-(2-chloro-4′-fluoro-[1,1′-biphenyl]-4-yl)-7-methyl-2,3-dioxo-1,2,3,4-tetrahydropyrido[2,3-b]pyrazine-6-sulfonamide (9)—synthesis according to Method 1 (M1)

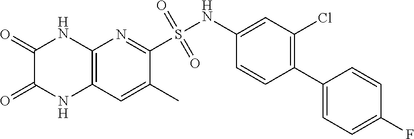 [SMILES: Cc1cc2[nH]c(=O)c(=O)[nH]c2nc1S(=O)(=O)Nc1ccc(-c2ccc(F)cc2)c(Cl)c1]

To a stirred solution of aniline 8 (200 mg, 0.727 mmol) in DCM (3 mL) at 0° C. was added pyridine (0.63 mL, 7.27 mmol). The resulting reaction mixture was stirred for 5 min, prior to the addition of sulfonyl chloride 7a (160 mg, 0.727 mmol) at 0° C. The mixture was stirred at r. t. for 16 h. The reaction was quenched with cold H₂O and the resulting solid was filtered and washed with water and Et₂O. The crude product was purified by prep-HPLC using NH₄CO₃/H₂O in MeCN as an eluent to yield the desired product 9 (40 mg, 12%) as an off white solid.

¹H NMR (400 MHz, DMSO-d₆) δ 12.57 (s, 1H), 12.15 (s, 1H), 7.41-7.34 (m, 4H), 7.26-7.22 (m, 4H), 2.57 (s, 3H).

MS (ES) C₂₀H₁₄ClFN₄O₄S requires: 460, found: 461 (M+H)⁺, 98% 2.1.3. Preparation of intermediate compounds of formula (C) and (D) 2,3-dioxo-7-(prop-1-en-2-yl)-N-(4-(trifluoromethoxy)phenyl)-1,2,3,4-tetrahydropyrido[2,3-b]pyrazine-6-sulfonamide (27-C)—synthesis according to Method 3 (M3)

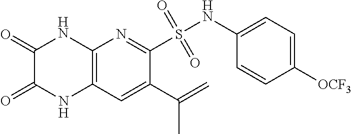 [SMILES: C=C(C)c1cc2[nH]c(=O)c(=O)[nH]c2nc1S(=O)(=O)Nc1ccc(OC(F)(F)F)cc1]

To a stirred solution of compound 25 (250 mg, 0.52 mmol) and 2-isopropenylboronic acid pinacol ester (438 mg, 2.60 mmol) in a mixture of 1,4-dioxane and water (3:1, 8 mL) at r. t. was added Cs₂CO₃ (677 mg, 2.08 mmol). The reaction was degassed for 15 min under a flow of N₂. After degassing, tBu₃PPdG2 (13 mg, 0.02 mmol) was added, and the reaction was stirred at 120° C. for 16 h. The mixture was quenched with H₂O (40 mL) and extracted with EtOAc (3×50 mL). The combined organic layer was dried over Na₂SO₄, filtered, and concentrated in vacuo. The crude was further purified by prep-HPLC NH₄HCO₃/H₂O in MeCN as an eluent to yield the desired product 27—C (25 mg, 11%) as an off-white solid.

¹H NMR (400 MHz, DMSO-d₆) δ 12.10-11.50 (m, 2H), 6.94 (s, 1H), 6.92-6.81 (m, 4H), 4.95 (s, 1H), 4.64 (s, 1H), 2.07 (s, 3H).

MS (ES) C₁₇H₁₃F₃N₄O₅S requires: 442, found: 443 (M+H)⁺, 99%

### 7-isopropyl-2,3-dioxo-N-(4-(trifluoromethoxy)phenyl)-1,2,3,4-tetrahydropyrido[2,3-b]pyrazine-6-sulfonamide (34-D)—synthesis according to Method 4 (M4)

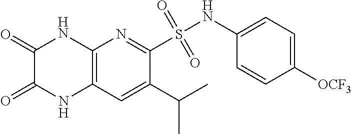 [SMILES: CC(C)c1cc2[nH]c(=O)c(=O)[nH]c2nc1S(=O)(=O)Nc1ccc(OC(F)(F)F)cc1]

To a stirred solution of compound 27 (20 mg, 0.04 mmol) in a mixture of MeOH and THF (1:1, 10 mL) was added Pt/C (20 mg) at RT, then the reaction was stirred at RT in parr-shaker reactor for 48 h. The reaction mixture was then filtered through celite, and the residue was washed with MeOH (2×50 mL). The filtrate was concentrated under in vacuo, and the resulting crude material was further purified by prep-HPLC using NH₄HCO₃/H₂O in MeCN as an eluent to yield the desired product 34-D (5 mg, 25%) as an off-white solid.

¹H NMR (400 MHz, DMSO-d₆) δ 12.52 (s, 1H), 12.08 (s, 1H), 10.77 (s, 1H), 7.42-7.22 (m, 6H), 3.84 (t, J=6.8 Hz, 1H), 1.17 (d, J=6.8 Hz, 6H).

MS (ES) C₁₇H₁₅F₃N₄O₅S requires: 444, found: 445 (M+H)⁺, 89%

Further compounds exemplifying the invention are described in Table 1.

When not otherwise specified, it should be assumed that methods M1, sometimes followed by M2 were used to yield the target compounds. The order of steps is highlighted in the ‘Synthetic Sequence’ column. Methods M3 and M4 were employed in the synthesis of compounds 27 and 34 from intermediate 25. A further deprotection or hydrolysis step might be required to obtain the final product, as would be recognized by a person skilled in the art. It should also be apparent to a person skilled in the art that reaction conditions such as temperature, dilution, reaction time or work-up procedures, including pH adjustment, are dependent on reaction partners and functional group compatibility and could vary from compound to compound.

TABLE 1
Compounds of formula (I) of the invention (A/B)

| | | Synthetic | | | | LC |
|---|---|---|---|---|---|---|
| | Structure | sequence | ¹H-NMR | (M + H)⁺ | (M − H)⁻ | purity |
| 9 | 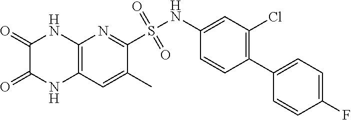 [SMILES: Cc1cc2[nH]c(=O)c(=O)[nH]c2nc1S(=O)(=O)Nc1ccc(-c2ccc(F)cc2)c(Cl)c1] | M2, M1 | ¹H NMR (400 MHz, DMSO- d₆) δ: 12.57 (s, 1H), 12.15 (s, 1H), 7.41-7.34 (m, 4H), 7.26- 7.22 (m, 4H), 2.57 (s, 3H). | | 459.0 | 99.36 |
| 10 | 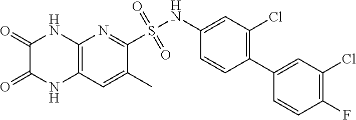 [SMILES: Cc1cc2[nH]c(=O)c(=O)[nH]c2nc1S(=O)(=O)Nc1ccc(-c2ccc(F)c(Cl)c2)c(Cl)c1] | M2, M1 | ¹H NMR (400 MHz, DMSO- d₆) δ: 12.56 (s, 1H), 12.18 (d, J = 5.6 Hz, 1H), 10.90 (s, 1H), 7.58, 7.56 (dd, J = 7.2, 2.0 Hz, | 494.9 | | 98.82 |
| | | | 1H), 7.46 (t, | | | |
| | | | J = 8.8 Hz, 1H), | | | |
| | | | 7.39-7.35 (m, | | | |
| | | | 3H), 7.30-7.25 | | | |
| | | | (m, 2H), 2.57 | | | |
| | | | (s, 3H). | | | |
| 11 | 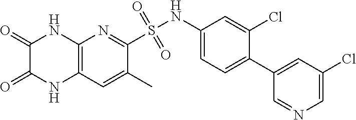 [SMILES: Cc1cc2[nH]c(=O)c(=O)[nH]c2nc1S(=O)(=O)Nc1ccc(-c2cncc(Cl)c2)c(Cl)c1] | M2, M1 | ¹H NMR (400 MHz, DMSO- d₆) δ: 12.65- 10.92 (m, 3H), 8.62 (d, J = 2.4 Hz, 1H), 8.53 (d, J = 1.6 Hz, 1H), 7.97 (s, | | 476.0 | 99.38 |
| | | | 1H), 7.37-7.28 | | | |
| | | | (m, 4H), 2.57 | | | |
| | | | (s, 3H). | | | |
| 12 | 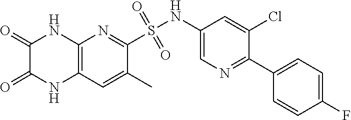 [SMILES: Cc1cc2[nH]c(=O)c(=O)[nH]c2nc1S(=O)(=O)Nc1cnc(-c2ccc(F)cc2)c(Cl)c1] | M2, M1 | ¹H NMR (400 MHz, DMSO- d₆) δ: 12.25- 11.33 (m, 2H), 7.90 (d, J = 2.4 Hz, 1H), 7.59- 7.55 (m, 2H), 7.27 (d, J = 2.0 | 462.1 | | 95.32 |
| | | | Hz, 1H), 7.18- | | | |
| | | | 7.14 (m, 2H), | | | |
| | | | 7.03 (s, 1H), | | | |
| | | | 2.50 (s, 3H). | | | |
| 13 | 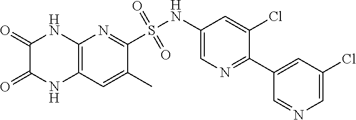 [SMILES: Cc1cc2[nH]c(=O)c(=O)[nH]c2nc1S(=O)(=O)Nc1cnc(-c2cncc(Cl)c2)c(Cl)c1] | M2, M1 | ¹H NMR (400 MHz, DMSO- d₆) δ: 12.61 (s, 1H), 12.21 (s, 1H), 11.29 (s, 1H), 8.80 (d, J = 1.6 Hz, 1H), 8.70 (d, | 479.08 | | 90.36 |
| | | | J = 2.4 Hz, 1H), | | | |
| | | | 8.66 (d, J = 2.4 | | | |
| | | | Hz, 1H), 8.18 | | | |
| | | | (t, J = 2.4 Hz, | | | |
| | | | 1H), 7.90 (d, | | | |
| | | | J = 2.4 Hz, | | | |
| | | | 1H), 7.41 (s, | | | |
| | | | 1H), 2.57 (s, | | | |
| | | | 3H). | | | |
| 14 | 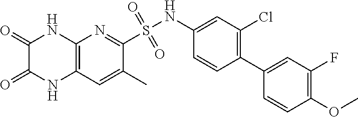 [SMILES: COc1ccc(-c2ccc(NS(=O)(=O)c3nc4[nH]c(=O)c(=O)[nH]c4cc3C)cc2Cl)cc1F] | M2, M1 | ¹H NMR (400 MHz, DMSO- d₆) δ: 12.57 (bs, 1H), 12.19 (bs, 1H), 10.87 (bs, 1H), 7.38 (s, 1H), 7.34 (s, 1H), 7.26-7.18 | 491.20 | | 99.71 |
| | | | (m, 4H), 7.13 | | | |
| | | | (dd, J = 8.4, 1.2 | | | |
| | | | Hz, 1H), 3.86 | | | |
| | | | (s, 3H), 2.57 (s, | | | |
| | | | 3H). | | | |
| 15 | 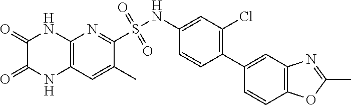 [SMILES: Cc1nc2cc(-c3ccc(NS(=O)(=O)c4nc5[nH]c(=O)c(=O)[nH]c5cc4C)cc3Cl)ccc2o1] | M2, M1 | ¹H NMR (400 MHz, DMSO- d₆) δ: 12.56- 12.19 (m, 2H), 10.89 (bs, 1H), 7.13 (d, J = 8.4 Hz, 1H), 7.59 | 498.27 | | 99.58 |
| | | | (d, J = 1.2 Hz, | | | |
| | | | 1H), 7.46-7.41 | | | |
| | | | (m, 5H), 2.62 | | | |
| | | | (s, 3H), 2.58 (s, | | | |
| | | | 3H). | | | |
| 16 | 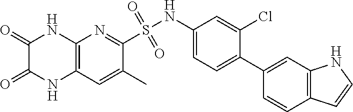 [SMILES: Cc1cc2[nH]c(=O)c(=O)[nH]c2nc1S(=O)(=O)Nc1ccc(-c2ccc3cc[nH]c3c2)c(Cl)c1] | M1, M2 | ¹H NMR (400 MHz, DMSO- d₆) δ: 12.14 (bs, 2H), 11.03 (s, 1H), 7.46 (d, J = 8.4 Hz, 1H), 7.31 (t, J = 2.8 | 482.20 | | 95.35 |
| | | | Hz, 1H), 7.28 | | | |
| | | | (s, 1H), 7.17 (s, | | | |
| | | | 1H), 6.93-6.90 | | | |
| | | | (m, 3H), 6.78 | | | |
| | | | (d, J = 8.4 Hz, | | | |
| | | | 1H), 6.39-6.38 | | | |
| | | | (m, 1H), 2.55 | | | |
| | | | (s, 3H). | | | |
| 17 | 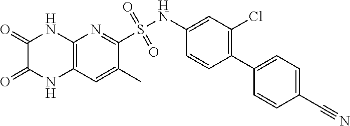 [SMILES: Cc1cc2[nH]c(=O)c(=O)[nH]c2nc1S(=O)(=O)Nc1ccc(-c2ccc(C#N)cc2)c(Cl)c1] | M2, M1 | ¹H NMR (400 MHz, DMSO- d₆) δ: 12.57 (bs, 1H), 12.19 (bs, 1H), 10.96 (bs, 1H), 7.89 (dd, J = 6.8, 2.0 Hz, 2H) 7.58 (dd, | 468.20 | | 97.44 |
| | | | J = 6.8, 2.0 Hz, | | | |
| | | | 2H), 7.39 (bs, | | | |
| | | | 2H), 7.31 (bs, | | | |
| | | | 2H), 2.57 (s, | | | |
| | | | 3H). | | | |
| 18 | 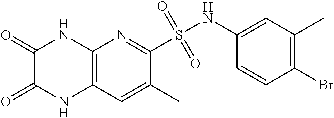 [SMILES: Cc1cc(NS(=O)(=O)c2nc3[nH]c(=O)c(=O)[nH]c3cc2C)ccc1Br] | M1 | ¹H NMR (400 MHz, DMSO- d₆) δ: 12.09 (bs, 3H), 7.34-7.32 (m, 2H), 7.15 (d, J = 1.2 Hz, 1H), 6.96 (dd, J = 8.4, 1.6 Hz, | 425.08 | | 99.87 |
| | | | 1H), 2.52 (s, | | | |
| | | | 3H), 2.23 (s, | | | |
| | | | 3H). | | | |
| 19 | 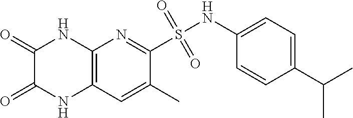 [SMILES: Cc1cc2[nH]c(=O)c(=O)[nH]c2nc1S(=O)(=O)Nc1ccc(C(C)C)cc1] | M1 | ¹H NMR (400 MHz, DMSO- d₆) δ: 12.14- 10.65 (m, 3H), 7.33 (s, 1H), 7.14 (d, J = 8.4 Hz, 2H), 7.07 (d, J = 8.4 Hz, | 375.10 | | 99.31 |
| | | | 2H), 2.79-2.56 | | | |
| | | | (m, 1H), 2.51 | | | |
| | | | (s, 3H), 1.12 (d, | | | |
| | | | J = 7.2 Hz, 6H). | | | |
| 20 | 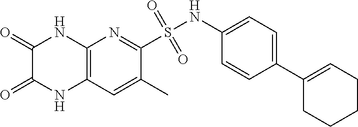 [SMILES: Cc1cc2[nH]c(=O)c(=O)[nH]c2nc1S(=O)(=O)Nc1ccc(C2=CCCCC2)cc1] | M2, M1 | ¹H NMR (400 MHz, DMSO- d₆) δ: 11.67 (bs, 3H), 7.29 (s, 1H), 7.20 (d, J = 8.4 Hz, 2H), 7.11 (d, J = 8.4 Hz, 2H), 6.02- | 413.27 | | 93.79 |
| | | | 6.00 (m, 1H), | | | |
| | | | 2.51 (s, 3H), | | | |
| | | | 2.27-2.25 (m, | | | |
| | | | 2H), 2.13-2.10 | | | |
| | | | (m, 2H), 1.67- | | | |
| | | | 1.64 (m, 2H), | | | |
| | | | 1.58-1.52 (m, | | | |
| | | | 2H). | | | |
| 21 | 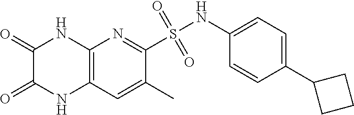 [SMILES: Cc1cc2[nH]c(=O)c(=O)[nH]c2nc1S(=O)(=O)Nc1ccc(C2CCC2)cc1] | M1 | ¹H NMR (400 MHz, DMSO- d₆) δ: 12.15- 10.56 (m, 2H), 7.30 (s, 1H), 7.13 (d, J = 8.4 Hz, 2H), 7.05 | 387.18 | | 99.85 |
| | | | (d, J = 8.4 Hz, | | | |
| | | | 2H), 3.41-3.35 | | | |
| | | | (m, 1H), 2.51 | | | |
| | | | (s, 3H), 2.23- | | | |
| | | | 2.17 (m, 2H), | | | |
| | | | 2.07-1.86 (m, | | | |
| | | | 3H), 1.78-1.71 | | | |
| | | | (m, 1H). | | | |
| 22 | 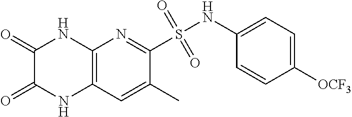 [SMILES: Cc1cc2[nH]c(=O)c(=O)[nH]c2nc1S(=O)(=O)Nc1ccc(OC(F)(F)F)cc1] | M1 | ¹H NMR (400 MHz, DMSO- d₆): δ 11.91 (broad s, 3H), 7.29 (s, 1H), 7.17-7.06 (m, 4H), 2.49 (s, 3H). | 417 | | 99% |
| 23 | 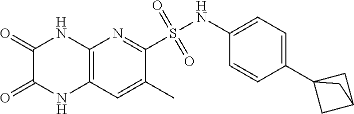 [SMILES: Cc1cc2[nH]c(=O)c(=O)[nH]c2nc1S(=O)(=O)Nc1ccc(C23CC(C2)C3)cc1] | M1 | ¹H NMR (400 MHz, DMSO- d₆) δ: 11.64 (brs, 1H), 7.22 (s, 1H), 7.06 (d, J = 8.4 Hz, 2H), 6.95 (d, | 399.32 | | 97.06 |
| | | | J = 8.4 Hz, 2H), | | | |
| | | | 2.50-2.47 (m, | | | |
| | | | 4H), 1.94 (s, | | | |
| | | | 6H). | | | |
| 24 | 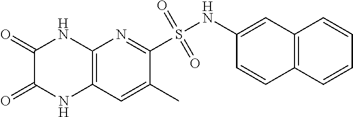 [SMILES: Cc1cc2[nH]c(=O)c(=O)[nH]c2nc1S(=O)(=O)Nc1ccc2ccccc2c1] | M1 | ¹H NMR (400 MHz, DMSO- d₆) δ: 11.73 (brs, 2H), 7.71- 7.64 (m, 3H), 7.54 (s, 1H), 7.37-7.30 (m, 2H), 7.27-7.22 | 383.22 | | 99.34 |
| | | | (m, 2H), 2.55 | | | |
| | | | (s, 3H). | | | |
| 25 | 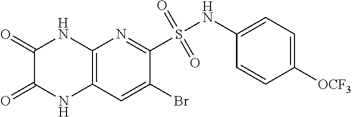 [SMILES: O=c1[nH]c2cc(Br)c(S(=O)(=O)Nc3ccc(OC(F)(F)F)cc3)nc2[nH]c1=O] | M1 | ¹H NMR (400 MHz, DMSO- d₆) δ: 11.89 (brs, 2H), 7.59 (s, 1H), 7.20 (d, J = 8.0 Hz, 2H), 7.11 (d, J = 8.0 Hz, 2H). | 481.06 | | 99.32 |
| 26 | 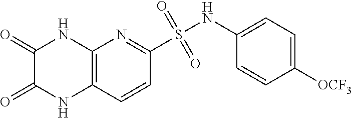 [SMILES: O=c1[nH]c2ccc(S(=O)(=O)Nc3ccc(OC(F)(F)F)cc3)nc2[nH]c1=O] | M1 | ¹H NMR (400 MHz, DMSO- d₆) δ: 12.04 (brs, 2H), 7.61 (d, J = 8.0 Hz, 1H), 7.47 (d, J = 8.0 Hz, 1H), 6.92-7.15 (m, | 403.16 | | 99.45 |
| | | | 4H). | | | |
| 27 | 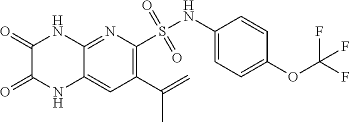 [SMILES: C=C(C)c1cc2[nH]c(=O)c(=O)[nH]c2nc1S(=O)(=O)Nc1ccc(OC(F)(F)F)cc1] | 25 + M3 | ¹H NMR (400 MHz, DMSO- d₆) δ: 12-10- 11.50 (m, 2H), 6.94 (s, 1H), 6.92-6.81 (m, 4H), 4.95 (s, 1H), 4.64 (s, | 443.15 | | 99.48 |
| | | | 1H), 2.07 (s, | | | |
| | | | 3H). | | | |
| 28 | 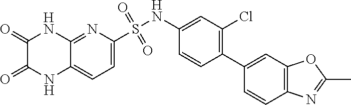 [SMILES: Cc1nc2ccc(-c3ccc(NS(=O)(=O)c4ccc5[nH]c(=O)c(=O)[nH]c5n4)cc3Cl)cc2o1] | M2, M1 | ¹H NMR (400 MHz, DMSO- d₆) δ: 12.61- 12.24 (m, 2H), 10.84 (bs, 1H), 7.78 (d, J = 8.0 Hz, 1H), 7.68 | 484.13 | | 98.72 |
| | | | (d, J = 8.8 Hz, | | | |
| | | | 1H), 7.60-7.58 | | | |
| | | | (m, 2H), 7.32- | | | |
| | | | 7.23 (m, 4H), | | | |
| | | | 2.62 (s, 3H). | | | |
| 29 | 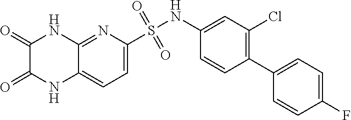 [SMILES: O=c1[nH]c2ccc(S(=O)(=O)Nc3ccc(-c4ccc(F)cc4)c(Cl)c3)nc2[nH]c1=O] | M2, M1 | ¹H NMR (400 MHz, DMSO- d₆) δ: 11.10- 8.80 (m, 3 H), 7.68 (d, J = 8.4 Hz, 1 H), 7.51 (d, J = 8.0 Hz, 1H), 7.38-7.34 | 447.19 | | 98.92 |
| | | | (m, 2 H), 7.23- | | | |
| | | | 7.18 (m, 2 H), | | | |
| | | | 7.10-7.08 (m, | | | |
| | | | 2H), 7.01 (d, | | | |
| | | | J = 8.4 Hz, 1H). | | | |
| 30 | 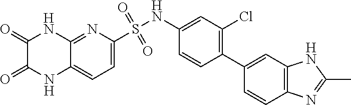 [SMILES: Cc1nc2ccc(-c3ccc(NS(=O)(=O)c4ccc5[nH]c(=O)c(=O)[nH]c5n4)cc3Cl)cc2[nH]1] | M2, M1 | ¹H NMR (400 MHz, DMSO- d₆) δ: 12.62 (bs, 2H), 12.24 (s, 1H), 10.79 (s, 1H), 7.78 (d, J = 8.0 Hz, 1H), | 483.18 | | 97.65 |
| | | | 7.60 (d, J = 8.4 | | | |
| | | | Hz, 1H), 7.48- | | | |
| | | | 7.23 (m, 5H), | | | |
| | | | 7.07 (d, J = 8.0 | | | |
| | | | Hz, 1H), 2.48 | | | |
| | | | (s, 3H). | | | |
| 31 | 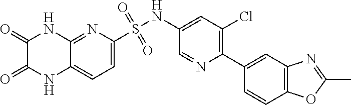 [SMILES: Cc1nc2cc(-c3ncc(NS(=O)(=O)c4ccc5[nH]c(=O)c(=O)[nH]c5n4)cc3Cl)ccc2o1] | M2, M1 | ¹H NMR (400 MHz, DMSO- d₆) δ: 12.64 (bs, 1H), 12.22 (bs, 1H), 11.12 (bs, 1H), 8.36 (s, 1H), 7.84 (s, | 485.09 | | 97.53 |
| | | | 1H), 7.77 (d, | | | |
| | | | J = 8.0 Hz, | | | |
| | | | 1H), 7.71-7.67 | | | |
| | | | (m, 2H), 7.59- | | | |
| | | | 7.55 (m, 2H), | | | |
| | | | 2.62 (s, 3H). | | | |
| 32 | 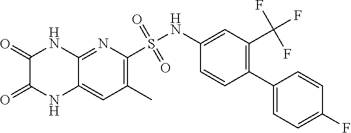 [SMILES: Cc1cc2[nH]c(=O)c(=O)[nH]c2nc1S(=O)(=O)Nc1ccc(-c2ccc(F)cc2)c(C(F)(F)F)c1] | M2, M1 | ¹H NMR (400 MHz, DMSO- d₆) δ: 12.53 (s, 1H), 12.19 (s, 1 H), 10.99 (s, 1H), 7.56 (br. s, 2H), 7.38 (s, 1H), 7.30-7.20 | 495.14 | | 99.01 |
| | | | (m, 5H), 2.57 | | | |
| | | | (s, 3H). | | | |
| 33 | 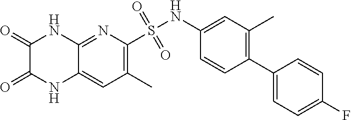 [SMILES: Cc1cc(NS(=O)(=O)c2nc3[nH]c(=O)c(=O)[nH]c3cc2C)ccc1-c1ccc(F)cc1] | M2, M1 | ¹H NMR (400 MHz, DMSO- d₆) δ: 12.53- 10.43 (m, 3 H), 7.36 (s, 1H), 7.30-7.27 (m, 2H), 7.20 (t, J = 9.2 Hz, | 441.17 | | 98.78 |
| | | | 2H), 7.13-7.09 | | | |
| | | | (m, 2H), 7.02 | | | |
| | | | (d, J = 8.4 Hz, | | | |
| | | | 1H), 2.55 (s, | | | |
| | | | 3H), 2.13 (s, | | | |
| | | | 3H). | | | |
| 34 | 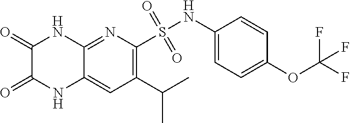 [SMILES: CC(C)c1cc2[nH]c(=O)c(=O)[nH]c2nc1S(=O)(=O)Nc1ccc(OC(F)(F)F)cc1] | 27 + M4 | ¹H NMR (400 MHz, DMSO- d₆) δ: 12.52 (s, 1H), 12.08 (s, 1H), 10.77 (s, 1H), 7.42-7.22 (m, 6H), 3.84 (t, J = 6.8 Hz, | 445.16 | | 89.37 |
| | | | 1H), 1.17 (d, | | | |
| | | | J = 6.8 Hz, 6H). | | | |
| 35 | 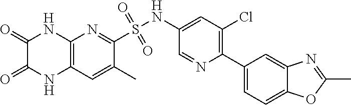 [SMILES: Cc1nc2cc(-c3ncc(NS(=O)(=O)c4nc5[nH]c(=O)c(=O)[nH]c5cc4C)cc3Cl)ccc2o1] | M2, M1 | ¹H NMR (400 MHz, DMSO- d₆) δ: 8.47 (d, J = 2.0 Hz, 1H), 7.89 (d, J = 2.4 Hz, 1H), 7.82 (d, J = 1.2 Hz, | 499.15 | | 97.06 |
| | | | 1H), 7.63-7.56 | | | |
| | | | (m, 2H), 7.45 | | | |
| | | | (s, 1H), 2.65 | | | |
| | | | (s, 6H). | | | |
| 36 | 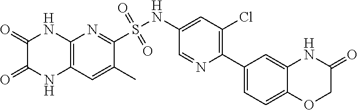 [SMILES: Cc1cc2[nH]c(=O)c(=O)[nH]c2nc1S(=O)(=O)Nc1cnc(-c2ccc3c(c2)NC(=O)CO3)c(Cl)c1] | M2, M1 | ¹H NMR (400 MHz, DMSO- d₆) δ: 12.60 (bs, 1H), 12.14 (bs, 1H), 11.13 (bs, 1H), 10.78 (s, 1H), 8.46 (bs, | 515.13 | | 99.00 |
| | | | 1H), 7.72 (s, | | | |
| | | | 1H), 7.37 (s, | | | |
| | | | 1H), 7.27-7.19 | | | |
| | | | (m, 2H), 6.98 | | | |
| | | | (d, J = 8.0, 1H), | | | |
| | | | 4.61 (s, 2H), | | | |
| | | | 2.56 (s, 3H). | | | |
| 37 | 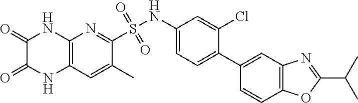 [SMILES: Cc1cc2[nH]c(=O)c(=O)[nH]c2nc1S(=O)(=O)Nc1ccc(-c2ccc3oc(C(C)C)nc3c2)c(Cl)c1] | M2, M1 | ¹H NMR (400 MHz, DMSO- d₆) δ: 12-24- 10.84 (m, 2H), 7.69 (d, J = 8.4 Hz, 1H), 7.64 (d, J = 1.2 Hz, | 526.22 | | 99.33 |
| | | | 1H), 7.37 (s, | | | |
| | | | 1H), 7.32-7.24 | | | |
| | | | (m, 4H), 3.29- | | | |
| | | | 3.23 (m, 1H), | | | |
| | | | 2.58 (s, 3H), | | | |
| | | | 1.38 (d, J = 6.8 | | | |
| | | | Hz, 6H). | | | |
| 38 | 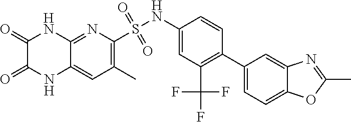 [SMILES: Cc1nc2cc(-c3ccc(NS(=O)(=O)c4nc5[nH]c(=O)c(=O)[nH]c5cc4C)cc3C(F)(F)F)ccc2o1] | M2, M1 | ¹H NMR (400 MHz, DMSO- d₆) δ: 12.52 (s, 1 H), 12.15 (s, 1H), 11.03 (s, 1H), 7.65 (d, J = 8.4 Hz,1H), 7.49 (d, J = 8.0 | 532.22 | | 99.87 |
| | | | Hz, 3H), 7.36 | | | |
| | | | (s, 1H), 7.22- | | | |
| | | | 7.17 (m, 2H), | | | |
| | | | 2.61 (s, 3H), | | | |
| | | | 2.58 (s, 3H). | | | |
| 39 | 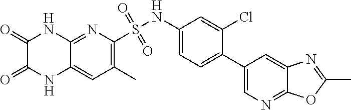 [SMILES: Cc1nc2cc(-c3ccc(NS(=O)(=O)c4nc5[nH]c(=O)c(=O)[nH]c5cc4C)cc3Cl)cnc2o1] | M2, M1 | ¹H NMR (400 MHz, DMSO- d₆) δ: 12.41 (s, 1H), 10.83 (s, 1H), 8.24 (d, J = 2.0 Hz, 1H), 8.10 (d, J = 1.6 | 499.16 | | 98.83 |
| | | | Hz, 1H), 7.36- | | | |
| | | | 7.24 (m, 4H), | | | |
| | | | 2.65 (s, 3H), | | | |
| | | | 2.57 (s, 3H). | | | |
| 40 | 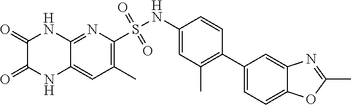 [SMILES: Cc1nc2cc(-c3ccc(NS(=O)(=O)c4nc5[nH]c(=O)c(=O)[nH]c5cc4C)cc3C)ccc2o1] | M2, M1 | ¹H NMR (400 MHz, DMSO- d₆) δ: 12.54- 10.51 (m, 3H), 7.64 (d, J = 8.4 Hz, 1H), 7.49 (d, J = 1.2 Hz, | 478.22 | | 99.93 |
| | | | 1H), 7.36 (s, | | | |
| | | | 1H), 7.19 (dd, | | | |
| | | | J = 8.0, 1.6 Hz, | | | |
| | | | 1H) 7.14-7.11 | | | |
| | | | (m, 2H), 7.07 | | | |
| | | | (d, J = 8.0 Hz, | | | |
| | | | 1H), 2.61 (s, | | | |
| | | | 3H), 2.56 (s, | | | |
| | | | 3H), 2.14 (s, | | | |
| | | | 3H). | | | |
| 41 | 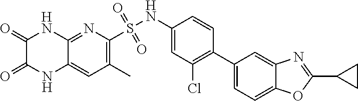 [SMILES: Cc1cc2[nH]c(=O)c(=O)[nH]c2nc1S(=O)(=O)Nc1ccc(-c2ccc3oc(C4CC4)nc3c2)c(Cl)c1] | M2, M1 | ¹H NMR (400 MHz, DMSO- d₆) δ: 12.57- 12.16 (m, 1H), 10.85 (s, 1H), 7.66 (d, J = 7.2 Hz, 1H), 7.54 | 524.15 | | 96.63 |
| | | | (d, J = 1.2 Hz, | | | |
| | | | 1H), 7.38-7.31 | | | |
| | | | (m, 2H), 7.27- | | | |
| | | | 7.24 (m, 3H), | | | |
| | | | 2.57 (s, 3H), | | | |
| | | | 2.30-2.25 (m, | | | |
| | | | 1H), 1.23-1.12 | | | |
| | | | (m, 4H). | | | |

### 3. Hemolysin Alpha-Based Ca²⁺-Influx Assay

The assay is based on the Fluo-4 NW Calcium Assay Kit (#F36205) from Thermo Scientific. Here the effect of hemolysin alpha from Staphylococcus aureus was monitored by loading non adherent U397 cells with the Ca²⁺-sensitive dye Fluo-4 hemolysin alpha addition leads to the formation of Ca²⁺ permissive pores in the membrane of the U397 cells which results in a dose dependent increase of fluorescence.

Briefly, the protocol described here was applied for screening and activity determination in a low-volume 384-well microtiter plate with cell culture treated surface. For high-throughput application in the 1536-well microtiter plate format, volumes of the reagent mixes were adjusted, maintaining the volumetric ratio.
a. Hemolysin alpha was diluted with PBS from its stock to a working concentration of 35 nM. U937 cells were diluted in assay buffer (HBBS, 20 mM HEPES) to 4000 cells/μl. 1× Fluo-4 NW dye loading solution was prepared according to manufacturer's specifications including 5 mM probenecid to reduce background fluorescence.
b. Chemical compounds were applied into empty assay plates using contact-free acoustic droplet-dispensing (Echo520® Labcyte Inc., Sunnyvale CA) from 10 mM compound stocks in 100% DMSO, to a final concentration of 10 μM or in serial dilution series of the required concentration range. Equal amounts of DMSO without any compound were added to control samples.
c. 5 μL of the U937 cells+5 μl of 1× Fluo-4 NW dye load were dispensed using a Multidrop® dispenser (Thermo Fisher Scientific, Waltham MA) into the wells of a microtiter plate. The plate was centrifuged at 1000 rpm and incubated at 37° C. for 30 min followed by an incubation step at r. t. for another 30 min.
d. The reaction was started by addition of 4 μl of hemolysin alpha to a final concentration of 10 nM followed by a centrifugation step for 1 min at 1000 rpm. No hemolysin alpha was added to negative control samples.
e. After incubation at room temperature for 4 h the generated signal was measured with an EnVision plate reader (Perkin Elmer, Waltham MA), using excitation at 485 nm and an emission at 535 nm.

### 4. LDH-Glo Cytotoxicity Assay

The LDH-Glo-Cytotoxicity Assay is a bioluminescent plate-based assay to quantify the release of cellular Lactate Dehydogenase (LDH) into the assay medium upon plasma membrane damage by hemolysin treatment of the cells. LDH in the supernatant reduces an added substrate to generate luciferin which is converted into a bioluminescent signal by the Ultra Glo Luciferase (Promega).

A549-Cells (DSMZ, #ACC107), that are used for the assay, are maintained in RPMI 1640 cell culture medium+glutamine (PAN Biotech GmbH, Aidenbach, Germany; #P04-22100; P04-05500) supplemented with 10% fetal calf serum (Capricorn, #FBS-11A) and are grown at 37° C., 5% CO₂.

For the LDH assay compounds or DMSO are prediluted at different concentrations in 15 μl cell culture medium RPMI 1640+5% FCS+10 mM HEPES in black μclear 384-well-plates (Greiner BioOne). Shortly afterwards 10 μl of 70 nM S. aureus Alpha hemolysin (IBT BIOSERVICES, #1401-002) was added to get a final assay concentration of 20 nM. After adding 10 μl of A549 cells (20.000 cells/well diluted in assay medium) the assay plates (total assay volume: 35 μl) were incubated for 5 h at 37° C./5% C₀₂ in humidified chambers in order to allow hemolysis.

As a positive internal control, we use the hemolysin antibody (IBT Bioservices, #0210-001) at a concentration range from 0.005-10 μg/ml and determine the IC₅₀ concentration. The standard IC₅₀ concentration for the antibody is approximately 50 ng/ml.

The determination of the LDH concentration was done after the 5 h incubation time according to the instructions of the One Glo Luminescent assay Kit (Promega, cat no. G7891). Shortly, 20 μl of cell culture supernatant were incubated in a separated black μclear 384-well plate at 25° C. for 20 min, mixed with 20 μl of the LDH reagent using an orbital shaker (1 min, 300 rpm) and further incubated for 5 min at 2° C. The reaction was stopped by addition of 10 μl stop-reagent, provided with the assay kit. Shortly afterwards the fluorescent signal was measured by Victor X5 plate reader (Perkin Elmer) using the filters 531 nm (extinction) and 590 nm (emission). EC₅₀ values were calculated with the software Excel Fit (IDBS, Guildford, UK) from 3-fold dilution series comprising at least 8 concentrations in duplicates.

### 5. Biological Activities of Compounds

Activities of compounds are listed in Table 3 together with compound number and IUPAC names. Biological activities are determined by two main assays with HIα-induced cell damage: hemolysin-α Ca2+-influx on U937 cells according to Example 3 and LDH-Glo Cytotoxicity Assay on A549 cells according to Example 4, and were grouped according to the following scheme:

| | <30 nM | 30 nM ≤ x < 100 nM | 100 nM ≤ x < 1 μM | 1 μM ≤ x < 3.5 μM |
|---|---|---|---|---|
| Ca²⁺-infux Assay | +++ | ++ | + | (+) |
| (IC-50) | | | | |
| | <30 nM | 30 nM ≤ x < 100 nM | 100 nM ≤ x < 1 μM | 1 μM ≤ x < 6 μM |
| A549-LDH Assay | +++ | ++ | + | (+) |
| (IC-50) | | | | |

TABLE 3
IUPAC chemical names and biological activities

| | | Ca²⁺-infux | A549-LDH |
|---|---|---|---|
| Ex | IUPAC Name | Assay activity | Assay activity |
| 9 | N-(2-chloro-4′-fluoro-[1,1′-biphenyl]-4-yl)-7-methyl-2,3- | +++ | + |
| | dioxo-1,2,3,4-tetrahydropyrido[2,3-b]pyrazine-6- | | |
| | sulfonamide | | |
| 10 | N-(2,3′-dichloro-4′-fluoro-[1,1′-biphenyl]-4-yl)-7-methyl- | ++ | (+) |
| | 2,3-dioxo-1,2,3,4-tetrahydropyrido[2,3-b]pyrazine-6- | | |
| | sulfonamide | | |
| 11 | N-(3-chloro-4-(5-chloropyridin-3-yl)phenyl)-7-methyl-2,3- | + | + |
| | dioxo-1,2,3,4-tetrahydropyrido[2,3-b]pyrazine-6- | | |
| | sulfonamide | | |
| 12 | N-(5-chloro-6-(4-fluorophenyl)pyridin-3-yl)-7-methyl-2,3- | ++ | (+) |
| | dioxo-1,2,3,4-tetrahydropyrido[2,3-b]pyrazine-6- | | |
| | sulfonamide | | |
| 13 | N-(3,5′-dichloro-[2,3′-bipyridin]-5-yl)-7-methyl-2,3-dioxo- | (+) | |
| | 1,2,3,4-tetrahydropyrido[2,3-b]pyrazine-6-sulfonamide | | |
| 14 | N-(2-chloro-3′-fluoro-4′-methoxy-[1,1′-biphenyl]-4-yl)-7- | ++ | + |
| | methyl-2,3-dioxo-1,2,3,4-tetrahydropyrido[2,3-b]pyrazine- | | |
| | 6-sulfonamide | | |
| 15 | N-(3-chloro-4-(2-methylbenzo[d]oxazol-5-yl)phenyl)-7- | +++ | ++ |
| | methyl-2,3-dioxo-1,2,3,4-tetrahydropyrido[2,3-b]pyrazine- | | |
| | 6-sulfonamide | | |
| 16 | N-(3-chloro-4-(1H-indol-6-yl)phenyl)-7-methyl-2,3-dioxo- | +++ | + |
| | 1,2,3,4-tetrahydropyrido[2,3-b]pyrazine-6-sulfonamide | | |
| 17 | N-(2-chloro-4′-cyano-[1,1′-biphenyl]-4-yl)-7-methyl-2,3- | ++ | + |
| | dioxo-1,2,3,4-tetrahydropyrido[2,3-b]pyrazine-6- | | |
| | sulfonamide | | |
| 18 | N-(4-bromo-3-methylphenyl)-7-methyl-2,3-dioxo-1,2,3,4- | + | + |
| | tetrahydropyrido[2,3-b]pyrazine-6-sulfonamide | | |
| 19 | N-(4-isopropylphenyl)-7-methyl-2,3-dioxo-1,2,3,4- | + | + |
| | tetrahydropyrido[2,3-b]pyrazine-6-sulfonamide | | |
| 20 | 7-methyl-2,3-dioxo-N-(2′,3′,4′,5′-tetrahydro-[1,1′- | + | + |
| | biphenyl]-4-yl)-1,2,3,4-tetrahydropyrido[2,3-b]pyrazine-6- | | |
| | sulfonamide | | |
| 21 | N-(4-cyclobutylphenyl)-7-methyl-2,3-dioxo-1,2,3,4- | +++ | ++ |
| | tetrahydropyrido[2,3-b]pyrazine-6-sulfonamide | | |
| 22 | 7-methyl-2,3-dioxo-N-(4-(trifluoromethoxy)phenyl)-1,2,3,4- | +++ | +++ |
| | tetrahydropyrido[2,3-b]pyrazine-6-sulfonamide | | |
| 23 | N-(4-(bicyclo[1.1.1]pentan-1-yl)phenyl)-7-methyl-2,3- | +++ | + |
| | dioxo-1,2,3,4-tetrahydropyrido[2,3-b]pyrazine-6- | | |
| | sulfonamide | | |
| 24 | 7-methyl-N-(naphthalen-2-yl)-2,3-dioxo-1,2,3,4- | + | + |
| | tetrahydropyrido[2,3-b]pyrazine-6-sulfonamide | | |
| 25 | 7-bromo-2,3-dioxo-N-(4-(trifluoromethoxy)phenyl)-1,2,3,4- | ++ | + |
| | tetrahydropyrido[2,3-b]pyrazine-6-sulfonamide | | |
| 26 | 2,3-dioxo-N-(4-(trifluoromethoxy)phenyl)-1,2,3,4- | + | + |
| | tetrahydropyrido[2,3-b]pyrazine-6-sulfonamide | | |
| 27 | 2,3-dioxo-7-(prop-1-en-2-yl)-N-(4- | +++ | +++ |
| | (trifluoromethoxy)phenyl)-1,2,3,4-tetrahydropyrido[2,3- | | |
| | b]pyrazine-6-sulfonamide | | |
| 28 | N-(3-chloro-4-(2-methylbenzo[d]oxazol-6-yl)phenyl)-2,3- | +++ | + |
| | dioxo-1,2,3,4-tetrahydropyrido[2,3-b]pyrazine-6- | | |
| | sulfonamide | | |
| 29 | N-(2-chloro-4′-fluoro-[1,1′-biphenyl]-4-yl)-2,3-dioxo- | + | (+) |
| | 1,2,3,4-tetrahydropyrido[2,3-b]pyrazine-6-sulfonamide | | |
| 30 | N-(3-chloro-4-(2-methyl-1H-benzo[d]imidazol-6-yl)phenyl)- | (+) | |
| | 2,3-dioxo-1,2,3,4-tetrahydropyrido[2,3-b]pyrazine-6- | | |
| | sulfonamide | | |
| 31 | N-(5-chloro-6-(2-methylbenzo[d]oxazol-5-yl)pyridin-3-yl)- | + | (+) |
| | 2,3-dioxo-1,2,3,4-tetrahydropyrido[2,3-b]pyrazine-6- | | |
| | sulfonamide | | |
| 32 | N-(4′-fluoro-2-(trifluoromethyl)-[1,1′-biphenyl]-4-yl)-7- | + | + |
| | methyl-2,3-dioxo-1,2,3,4-tetrahydropyrido[2,3-b]pyrazine- | | |
| | 6-sulfonamide | | |
| 33 | N-(4′-fluoro-2-methyl-[1,1′-biphenyl]-4-yl)-7-methyl-2,3- | + | ++ |
| | dioxo-1,2,3,4-tetrahydropyrido[2,3-b]pyrazine-6- | | |
| | sulfonamide | | |
| 34 | 7-isopropyl-2,3-dioxo-N-(4-(trifluoromethoxy)phenyl)- | ++ | + |
| | 1,2,3,4-tetrahydropyrido[2,3-b]pyrazine-6-sulfonamide | | |
| 35 | N-(5-chloro-6-(2-methylbenzo[d]oxazol-5-yl)pyridin-3-yl)-7- | +++ | + |
| | methyl-2,3-dioxo-1,2,3,4-tetrahydropyrido[2,3-b]pyrazine- | | |
| | 6-sulfonamide | | |
| 36 | N-(5-chloro-6-(3-oxo-3,4-dihydro-2H-benzo[b][1,4]oxazin- | ++ | (+) |
| | 6-yl)pyridin-3-yl)-7-methyl-2,3-dioxo-1,2,3,4- | | |
| | tetrahydropyrido[2,3-b]pyrazine-6-sulfonamide | | |
| 37 | N-(3-chloro-4-(2-isopropylbenzo[d]oxazol-5-yl)phenyl)-7- | +++ | + |
| | methyl-2,3-dioxo-1,2,3,4-tetrahydropyrido[2,3-b]pyrazine- | | |
| | 6-sulfonamide | | |
| 38 | 7-methyl-N-(4-(2-methylbenzo[d]oxazol-5-yl)-3- | +++ | + |
| | (trifluoromethyl)phenyl)-2,3-dioxo-1,2,3,4- | | |
| | tetrahydropyrido[2,3-b]pyrazine-6-sulfonamide | | |
| 39 | N-(3-chloro-4-(2-methyloxazolo[5,4-b]pyridin-6-yl)phenyl)- | +++ | ++ |
| | 7-methyl-2,3-dioxo-1,2,3,4-tetrahydropyrido[2,3- | | |
| | b]pyrazine-6-sulfonamide | | |
| 40 | 7-methyl-N-(3-methyl-4-(2-methylbenzo[d]oxazol-5- | +++ | ++ |
| | yl)phenyl)-2,3-dioxo-1,2,3,4-tetrahydropyrido[2,3- | | |
| | b]pyrazine-6-sulfonamide | | |
| 41 | N-(3-chloro-4-(2-cyclopropylbenzo[d]oxazol-5-yl)phenyl)-7- | +++ | ++ |
| | methyl-2,3-dioxo-1,2,3,4-tetrahydropyrido[2,3-b]pyrazine- | | |
| | 6-sulfonamide | | |

1. A compound of formula (I): 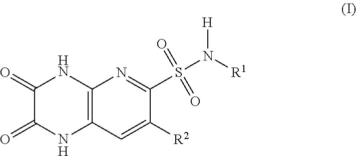
wherein
R² is hydrogen, halogen, OH, NO₂, CN or NH₂; or a C₁₋₄ alkyl group, a C₂₋₄ alkenyl group, a C₂₋₄ alkynyl group, a C₃₋₅ cycloalkyl group, an —O—C₃₋₅ cycloalkyl group, a C₄₋₈ alkylcycloalkyl group, or a C₁₋₄ heteroalkyl group; and
R¹ is an optionally substituted phenyl group; an optionally substituted naphthyl group; an optionally substituted heteroaryl group containing 1 or 2 rings and 5 to 10 ring atoms selected from O, S, N and C; an optionally substituted cycloalkyl aryl group comprising a phenyl group and a cycloalkyl group containing 5 or 6 ring atoms; an optionally substituted heterocycloalkyl aryl group comprising a phenyl group and a heterocycloalkyl group containing 5 or 6 ring atoms selected from O, S, B, N and C; an optionally substituted cycloalkyl heteroaryl group comprising a heteroaryl group comprising 5 or 6 ring atoms selected from O, S, N and C and a cycloalkyl group containing 5 or 6 ring atoms; or an optionally substituted heterocycloalkyl heteroaryl group comprising a heteroaryl group comprising 5 or 6 ring atoms selected from O, S, N and C and a heterocycloalkyl group containing 5 or 6 ring atoms selected from O, S, N and C; or an optionally substituted cycloalkyl group containing 1 or 2 rings and 3 to 10 ring atoms;
or a solvate, a hydrate or a salt thereof.

2. A compound according to claim 1, wherein R² is H, F, Cl, Br, a methyl group, an ethyl group, an iso-propyl group, a —C(CH₃)=CH₂ group, a—OCD₃ group, a —CF₃ group, a methoxy group, a —O—CF₃ group, a cyclopropyl group, a CN group, a CD₃ group, a —CHF₂ group, a —CH₂F group, a —CH₂OH group, a —NHMe group, an —O-cyclopropyl group, an —O—CH₂CF₃ group, an ethoxy group, an —NHCH₂CH₂OH group, or a —NMe₂ group.

3. A compound according to claim 1, wherein R² is a methyl group.

4. A compound according to any one of the preceding claims, wherein R¹ is an optionally substituted phenyl group; an optionally substituted naphthyl group; or an optionally substituted heteroaryl group containing 1 or 2 rings and 5 to 10 ring atoms selected from O, S, N and C.

5. A compound according to any one of the preceding claims 1 to 3, wherein R¹ has the following formula: 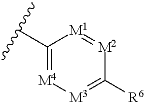
wherein
M¹ is N or CR⁷; M² is N or CR⁵; M³ is N or CR^{5a}; and M⁴ is N or CR^{7a};
R⁵, R^{5a}, R⁷ and R^{7a} are independently selected from hydrogen, halogen, CN, a C₁₋₄ alkyl group, a C₂₋₄ alkenyl group, a C₂₋₄ alkynyl group, or a C₁₋₄ heteroalkyl group; and
R⁶ is halogen, CN, an alkyl group, an alkenyl group, an alkynyl group, a heteroalkyl group, a cycloalkyl group, a heterocycloalkyl group, an alkylcycloalkyl group, a heteroalkylcycloalkyl group, an aryl group, a heteroaryl group, an aralkyl group or a heteroaralkyl group; all of which groups may optionally be substituted; or
R⁶ is a group of formula —OR^{6a} or —NHR^{6a}, wherein R^{6a} is a cycloalkyl group, a heterocycloalkyl group, an alkylcycloalkyl group, a heteroalkylcycloalkyl group, an aryl group, a heteroaryl group, an aralkyl group or a heteroaralkyl group; all of which groups may optionally be substituted; or
R⁵ and R⁶ together are part of an optionally substituted phenyl group, an optionally substituted heteroaryl group containing 5 or 6 ring atoms selected from O, S, N and C, an optionally substituted cycloalkyl group containing 5 or 6 ring atoms or an optionally substituted heterocycloalkyl group containing 5 or 6 ring atoms selected from O, S, B, N and C.

6. A compound according to claim 5, wherein R⁷ is hydrogen or methyl; preferably hydrogen.

7. A compound according to claim 5 or 6, wherein R^{7a} is hydrogen.

8. A compound according to any one of the preceding claims 1 to 3, wherein R¹ has the following formula: 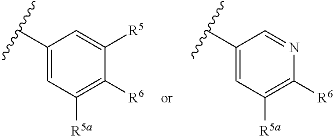
wherein
R⁵ and R^{5a} are independently selected from hydrogen, halogen, CN, a C₁₋₄ alkyl group, a C₂₋₄ alkenyl group, a C₂₋₄ alkynyl group, or a C₁₋₄ heteroalkyl group; and
R⁶ is halogen, CN, an alkyl group, an alkenyl group, an alkynyl group, a heteroalkyl group, a cycloalkyl group, a heterocycloalkyl group, an alkylcycloalkyl group, a heteroalkylcycloalkyl group, an aryl group, a heteroaryl group, an aralkyl group or a heteroaralkyl group; all of which groups may optionally be substituted.

9. A compound according to any one of the preceding claims 5 to 8, wherein R⁵ is hydrogen or methyl; especially hydrogen.

10. A compound according to any one of the preceding claims 5 to 9, wherein R^{5a} is hydrogen, Cl, Br, —CN, methyl, methoxy, —CF₃, —OCF₃, —NMe₂, —C≡CH, or —SO₂Me; especially hydrogen, Cl or methyl.

11. A compound according to any one of the preceding claims 5 to 10, wherein R⁶ is F, Cl, Br, CN, a C₁₋₆ alkyl group, a C₂₋₆ alkenyl group, a C₂₋₆ alkynyl group, a C₁₋₆ heteroalkyl group, an optionally substituted C₃₋₈ cycloalkyl group, an optionally substituted heterocycloalkyl group containing one or two rings and from 3 to 10 ring atoms selected from O, S, C and N, an optionally substituted phenyl group, an optionally substituted —CH₂-phenyl group, an optionally substituted heteroaryl group containing 5 or 6 to 10 ring atoms selected from O, S, N and C or an optionally substituted heterocycloalkyl aryl group comprising a phenyl group and a heterocycloalkyl group containing 4, 5 or 6 ring atoms selected from O, S, N and C.

12. A compound according to any one of the preceding claims 5 to 10, wherein R⁶ is an optionally substituted phenyl group; or an optionally substituted heteroaryl group containing 5 or 6 to 10 ring atoms selected from O, S, N and C; or an optionally substituted heterocycloalkyl aryl group comprising a phenyl group and a heterocycloalkyl group containing 4, 5 or 6 ring atoms selected from O, S, N and C; or a —O—CF₃ group.

13. A compound according to any one of the preceding claims 5 to 12, wherein R⁶ is unsubstituted or substituted by 1, 2 or 3 substituents that are independently selected from halogen, CN, OH, NH₂, ═O, CONH₂, COOH, a C₁₋₄ alkyl group, a C₂₋₄ alkenyl group, a C₂₋₄ alkynyl group, a C₁₋₄ heteroalkyl group, a C₃₋₇ cycloalkyl group, an —O—C₃₋₇ cycloalkyl group or a heterocycloalkyl group containing from 3 to 7 ring atoms selected from O, S, C and N;
especially wherein R⁶ is unsubstituted or substituted by 1, 2 or 3 substituents that are independently selected from F, Cl, Br, CN, COOH, OH, NH₂, a —CF₃ group, a—CD₃ group, a —NMe₂ group, a methyl group, an iso-propyl group, a cyclopropyl group, a C₂₋₄ alkynyl group, a methoxy group, a C₁₋₄ heteroalkyl group, or a —CONH₂ group.

14. Pharmaceutical composition comprising a compound according to anyone of the preceding claims and optionally one or more carrier substances and/or one or more adjuvants.

15. Compound according to any one of claims 1 to 13 or pharmaceutical composition according to claim 14 for use in the prophylaxis, decolonization and treatment of a Staphylococcus aureus infection; especially for use in the prophylaxis and treatment of pneumonia caused by Staphylococcus aureus.
